(19) United States
(12) Patent Application Publication
(10) Pub. No.: US 2021/0393759 A1
(43) Pub. Date: Dec. 23, 2021

(54) METHODS OF TREATING CANCER USING HETEROARYL-BIPHENYL AMIDE DERIVATIVES

(71) Applicant: CHEMOCENTRYX, INC., San Carlos, CA (US)

(72) Inventors: Shijie LI, Los Altos, CA (US); Marta VILALTA-COLOMER, Mountain View, CA (US); Yibin ZENG, Foster City, CA (US); Penglie ZHANG, Foster City, CA (US)

(21) Appl. No.: 17/354,003

(22) Filed: Jun. 22, 2021

Related U.S. Application Data
(60) Provisional application No. 63/042,807, filed on Jun. 23, 2020.

(51) Int. Cl. A61K 39/00; C07K 14/705; A61P 35/00

(52) U.S. Cl. CPC A61K 39/001111; A61P 35/00; C07K 14/70521

(57) ABSTRACT

Provided herein are methods of treating certain cancers comprising administering to the subject in need there of an effective amount of a compound of Formula (I)

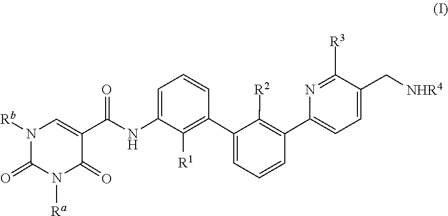

including stereoisomers and pharmaceutically acceptable salts thereof, wherein R¹, R², R³, R⁴, R^{a}, and R^{b} are as defined herein.

### CROSS-REFERENCES TO RELATED APPLICATIONS

This application claims the benefit of priority under 35 U.S.C. § 119(e) to U.S. Provisional Application Ser. No. 63/042,807 filed Jun. 23, 2020, the disclosure of which is incorporated herein by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE DISCLOSURE

Programmed cell death protein-1 (PD-1) is a member of the CD28 superfamily that delivers negative signals upon interaction with its two ligands, PD-L1 or PD-L2. PD-1 and its ligands are broadly expressed and exert a wide range of immunoregulatory roles in T cell activation and tolerance. PD-1 and its ligands are involved in attenuating infectious immunity and tumor immunity, and facilitating chronic infection and tumor progression.

Modulation of the PD-1 pathway has therapeutic potential in various human diseases (Hyun-Tak Jin et al., Curr Top Microbiol Immunol. (2011); 350:17-37). Blockade of the PD-1 pathway has become an attractive target in cancer therapy. Therapeutic antibodies that block the programmed cell death protein-1 (PD-1) immune checkpoint pathway prevent T-cell down regulation and promote immune responses against cancer. Several PD-1 pathway inhibitors have shown robust activity in various phases of clinical trials (RD Harvey, Clinical Pharmacology and Therapeutics (2014); 96(2), 214-223).

Agents that block the interaction of PD-L1 with either PD-1 or CD80 are desired. Some antibodies have been developed and commercialized. A few patent applications disclosing non-peptidic small molecules have been published (WO 2015/160641, WO 2015/034820, and WO 2017/066227 and WO2018/009505 from BMS; WO 2015/033299 and WO 2015/033301 from Aurigene; WO 2017/070089, US 2017/0145025, WO 2017/106634, US2017/0174679, WO2017/192961, WO2017/222976, WO2017/205464, WO2017/112730, WO2017/041899 and WO2018/013789 from Incyte, WO2018/006795 from Maxinovel and WO2018/005374 from us, ChemoCentryx). However there is still a need for alternative compounds such as small molecules as inhibitors of PD-L1, and which may have advantageous characteristics in term of oral administration, increased tumor penetration, stability, bioavailability, therapeutic index, and toxicity.

### BRIEF SUMMARY OF THE DISCLOSURE

In some aspects, provided herein are methods of treating a cancer comprising administering to a subject in need thereof an effective amount of a compound of Formula (I):

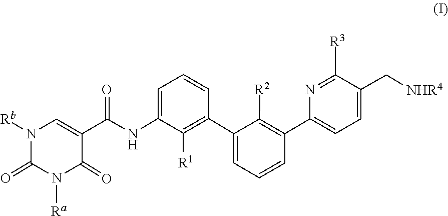

or a pharmaceutically acceptable salt thereof, wherein R¹, R², R³, R⁴, R^{a}, and R^{b} are as described herein.

In some embodiments the cancer is selected from the group consisting of colon cancer, renal cancer, colorectal cancer, gastric cancer, bladder cancer, melanoma, non-small cell lung cancer, Merkel cell carcinoma, liver cancer, breast cancer, and cancer of the head or neck.

### BRIEF DESCRIPTION OF THE DRAWINGS

FIG. 1A-B plots the PD1/PD-L1 binding ELISA data (upper panel) and PD-1/PD-L1 Blockade Cell-based assay data (lower panel) for Compounds 2.001 (A) and 2.002 (B).

FIG. 2A-C shows how Compound 2.001 promotes an allogenic immune response of human T cells in an ex vivo mixed lymphocyte reaction (MLR) assay; responses of T cells from three separate donors are shown: Donor #1 (A), Donor #2 (B), and Donor #3 (C).

FIG. 3A-C shows how Compound 2.002 promotes an allogenic immune response of human T cells in an ex vivo mixed lymphocyte reaction (MLR) assay; responses of T cells from three separate donors are shown: Donor #1 (A), Donor #2 (B), and Donor #3 (C).

FIG. 4A-B illustrate PBMC-mediated tumor cell killing of Compound 2.002 (A, left most columns), Compound 2.001 (A, middle columns), and a control compound (A, right most columns). Additional control experiments used an anti-PD-L1 antibody (Durvalumab) (B, left most columns), and antibody isotype (B, right most columns).

FIG. 5 shows that Compounds 2.001 and 2.002 induce PD-L1 dimerization, whereas the anti-PD-L1 antibody and tested controls do not.

FIG. 6 shows surface levels of PD-L1 at 4° C. (lower panel) and 37° C. (upper panel) under various test conditions. This figure demonstrates that Compound 2.001 and 2.002 lower the surface PD-L1 levels specifically at 37° C., suggesting PD-L1 internalization.

FIG. 7 MC38-hPD-L1 Tumor Model for Assessing Human PD-L1 Inhibitors in vivo. The engineered MC38-hPD-L1 cells are suitable for assessing the effects of human PD-L1 specific inhibitors in vivo: hPD-L1 and mPD-L1 bind to mPD-1 with similar affinity; the current hPD-L1 inhibitors block hPD-L1 interaction with hPD-1 or mPD-1 with similar Potency (data not shown). MC38-hPD-L1 cells induce tumor growth in mice.

FIG. 8A-C illustrates Compound 2.002 mediated tumor growth suppression in a dose-dependent manner in a MC38-hPD-L1 tumor model. (A) Plots the tumor volume v. the days after tumor implantation; (B) Plots the average tumor weight after 35 days; (C) Plots plasma compound concentrations at trough after 3 days of dosing

FIG. 9A-C plots the tumor size at the indicated number of days for vehicle treatment (filled circles) and API (anti-PD-L1 antibody or the indicated compound, filled squares). The APIs tested were Compound 2.001 (A), Compound 2.003 (B) and anti-PD-L1 antibody (C). The upper pane plots the average tumor size for each treatment group, while the lower pane plots the tumor size of each mouse in the treatment group.

FIG. 10A-B plots the trough plasma concentration of Compound 2.001 (A) and Compound 2.003 (B) 12 hour post dose, after 6 days of dosing, in the mouse model described in Biological Example 2.

FIG. 11 shows human PD-L1 staining of cells when treated with anti-PD-L1 antibody (Durvalumab), isotype antibody, Compound 2.001, and vehicle. The detection antibody of PD-L1 used in this analysis is blocked by Compound 2.001 binding to PD-L1. This figure demonstrates that Compound 2.001 treated MC38-hPD-L1 tumors have a near complete occupancy of Compound 2.001.

FIG. 12 shows how various treatment conditions alter the amount of tumor infiltrating immune cells in the MC38-HPD-L1 tumor model. Lower panel plots the amount of CD8+ T cells measured; middle panel plots the amount of CD4⁺ T cells measured; and upper panel plots the amount of CD8⁺ and CD4⁺ T cells measured.

### DETAILED DESCRIPTION OF THE DISCLOSURE

### I. General

The present disclosure provides methods for treating particular cancers using compounds of Formula (I). The claimed compounds possess robust anti-tumor properties, possessing a high affinity for PD-L1. When administered, these compounds effectively disrupt PD-1/PD-L1 signaling, and, in some embodiments, induce dimerization and internalization of PD-L1 on cancer cells.

The development of PD-1/PD-L1 small molecule modulators has been hindered by the need to balance a variety of factors including: PD-1/PD-L1 affinity, hydrophobicity/hydrophilcity of the compounds, biologic clearance rate, and antitarget activity (e.g., CYP and hERG inhibition). Indeed, to date, there are no approved PD-1/PD-L1 inhibitors for oral administration.

In contrast to IV drug formulations, bioavailability of an orally administered compound requires, among other things, gastric absorption and resistance to significant degradation through portal circulation to the liver (so called “first-pass metabolism”). In some embodiments, the methods described herein provide PD-1/PD-L1 modulators that are unexpectedly suitable for oral administration in the treatment of certain cancers. The compounds in the described methods do not require an extremely high concentration of compound in the blood; instead, these compounds can elicit their anti-tumor effects in the ng/mL range.

### II. Abbreviation and Definitions

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” may mean values that are within an order of magnitude, preferably within 5-fold and more preferably within 2-fold of a given value.

Numerical quantities given herein are approximate unless stated otherwise, meaning that the term “about” or “approximately” can be inferred when not expressly stated.

The term “alkyl”, by itself or as part of another substituent, means, unless otherwise stated, a straight or branched chain hydrocarbon group, having the number of carbon atoms designated (i.e. C₁₋₈ means one to eight carbons). Examples of alkyl groups include methyl, ethyl, n-propyl, isopropyl, n-butyl, t-butyl, isobutyl, .sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of alkenyl groups include vinyl, 2-propenyl, crotyl, 2-isopentenyl, 2-(butadienyl), 2,4-pentadienyl and 3-(1,4-pentadienyl). Examples of alkynyl groups include ethynyl, 1- and 3-propynyl, 3-butynyl, and the higher homologs and isomers. The term “cycloalkyl” refers to hydrocarbon rings having the indicated number of ring atoms (e.g., C₃₋₆ cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc. The bicyclic or polycyclic rings may be fused, bridged, spiro or a combination thereof. The term “heterocycloalkyl” or “heterocyclyl” refers to a cycloalkyl group that contain from one to five heteroatoms selected from N, O, and S, wherein the nitrogen and sulfur atoms are optionally oxidized, and the nitrogen atom(s) are optionally quaternized. The heterocycloalkyl may be a monocyclic, a bicyclic or a polycyclic ring system. The bicyclic or polycyclic rings may be fused, bridged, spiro or a combination thereof. It is understood that the recitation for C₄₋₁₂ heterocyclyl, refers to a group having from 4 to 12 ring members where at least one of the ring members is a heteroatom. Non limiting examples of heterocycloalkyl groups include pyrrolidine, imidazolidine, pyrazolidine, butyrolactam, valerolactam, imidazolidinone, tetrazolone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrahydrothiophene, quinuclidine, and the like. A heterocycloalkyl group can be attached to the remainder of the molecule through a ring carbon or a heteroatom.

The term “alkylene” by itself or as part of another substituent means a divalent group derived from an alkane, as exemplified by —CH₂CH₂CH₂CH₂—. An alkylene group can be linear or branched. An examples of the latter are —CH₂C(CH₃)₂CH₂—, —CH₂C(CH₃)₂— or —CH(CH₃)CH₂CH₂—. Typically, an alkyl (or alkylene) group will have from 1 to 12 carbon atoms, with those groups having 8 or fewer carbon atoms being preferred in the present disclosure. Similarly, “alkenylene” and “alkynylene” refer to the unsaturated forms of “alkylene” having double or triple bonds, respectively.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is meant to include trifluoromethyl, 2,2,2-trifluoroethyl, 4-chlorobutyl, 3-bromopropyl, and the like.

The term “hydroxyalkyl” or “alkyl-OH” refers to an alkyl group, as defined above, where at least one (and up to three) of the hydrogen atoms is replaced with a hydroxy group. As for the alkyl group, hydroxyalkyl groups can have any suitable number of carbon atoms, such as C₁₋₆. Exemplary hydroxyalkyl groups include, but are not limited to, hydroxymethyl, hydroxyethyl (where the hydroxy is in the 1- or 2-position), hydroxypropyl (where the hydroxy is in the 1-, 2- or 3-position), and 2,3-dihydroxypropyl.

The term “aryl” means, unless otherwise stated, a polyunsaturated, typically aromatic, hydrocarbon group which can be a single ring or multiple rings (up to three rings) which are fused together or linked covalently. The term “heteroaryl” refers to aryl groups (or rings) that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom. It is understood that the recitation for C₅₋₁₀ heteroaryl, refers to a heteroaryl moiety having from 5 to 10 ring members where at least one of the ring members is a heteroatom. Non-limiting examples of aryl groups include phenyl, naphthyl and biphenyl, while non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl and the like. Substituents for each of the above noted aryl and heteroaryl ring systems are selected from the group of acceptable substituents described below.

The term “carbocyclic ring,” “carbocyclic” or “carbocyclyl” refers to cyclic moieties with only carbon atoms as ring vertices. Carbocyclic ring moieties are saturated or unsaturated and can be aromatic. Generally, carbocyclic moieties have from 3 to 10 ring members. Carbocyclic moieties with multiple ring structure (e.g. bicyclic) can include a cycloalkyl ring fused to an aromatic ring (e.g. 1,2,3,4-tetrahydronaphthalene). Thus, carbocyclic rings include cyclopentyl, cyclohexenyl, naphthyl, and 1,2,3,4-tetrahydronaphthyl. The term “heterocyclic ring” refers to both “heterocycloalkyl” and “heteroaryl” moieties. Thus, heterocyclic rings are saturated or unsaturated and can be aromatic. Generally, heterocyclic rings are 4 to 10 ring members and include piperidinyl, tetrazinyl, pyrazolyl and indolyl.

When any of the above terms (e.g., “alkyl,” “aryl” and “heteroaryl”) are referred to as ‘substituted’ without further notation on the substituents, the substituted forms of the indicated group will be as provided below.

Substituents for the alkyl groups (including those groups often referred to as alkylene, alkenyl, alkynyl and cycloalkyl) can be a variety of groups selected from: -halogen, —OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —CN and —NO₂ in a number ranging from zero to (2 m′+1), where m′ is the total number of carbon atoms in such group. R′, R″ and R′″ each independently refer to hydrogen, unsubstituted C₁₋₈ alkyl, unsubstituted heteroalkyl, unsubstituted aryl, aryl substituted with 1-3 halogens, unsubstituted C₁₋₈ alkyl, C₁₋₈ alkoxy or C₁₋₈ thioalkoxy groups, or unsubstituted aryl-C₁₋₄ alkyl groups. When R′ and R″ are attached to the same nitrogen atom, they can be combined with the nitrogen atom to form a 3-, 4-, 5-, 6-, or 7-membered ring. For example, —NR′R″ is meant to include 1-pyrrolidinyl and 4-morpholinyl. The term “acyl” as used by itself or as part of another group refers to an alkyl group wherein two substitutents on the carbon that is closest to the point of attachment for the group is replaced with the substitutent ═O (e.g., —C(O)CH₃, —C(O)CH₂CH₂OR′ and the like).

Similarly, substituents for the aryl and heteroaryl groups are varied and are generally selected from: -halogen, —OR′, —OC(O)R′, —NR′R″, —SR′, —R′, —CN, —NO₂, —CO₂R′, —CONR′R″, —C(O)R′, —OC(O)NR′R″, —NR″C(O)R′, —NR″C(O)₂R′, —NR′—C(O)NR″R′″, —NH—C(NH₂)═NH, —NR′C(NH₂)═NH, —NH—C(NH₂)═NR′, —S(O)R′, —S(O)₂R′, —S(O)₂NR′R″, —NR'S(O)₂R″, —N₃, perfluoro(C₁-C₄)alkoxy, and perfluoro(C₁-C₄)alkyl, in a number ranging from zero to the total number of open valences on the aromatic ring system; and where R′, R″ and R′″ are independently selected from hydrogen, C₁₋₈ alkyl, C₃₋₆ cycloalkyl, C₂₋₈ alkenyl, C₂₋₈ alkynyl, unsubstituted aryl and heteroaryl, (unsubstituted aryl)-C₁₋₄ alkyl, and unsubstituted aryloxy-C₁₋₄ alkyl. Other suitable substituents include each of the above aryl substituents attached to a ring atom by an alkylene tether of from 1-4 carbon atoms.

Two of the substituents on adjacent atoms of the aryl or heteroaryl ring may optionally be replaced with a substituent of the formula -T-C(O)—(CH₂)_{q}—U—, wherein T and U are independently —NH—, —O—, —CH₂— or a single bond, and q is an integer of from 0 to 2.

Alternatively, two of the substituents on adjacent atoms of the aryl or heteroaryl ring may optionally be replaced with a substituent of the formula -A-(CH₂)ᵣ—B—, wherein A and B are independently —CH₂—, —O—, —NH—, —S—, —S(O)—, —S(O)₂—, —S(O)₂NR′— or a single bond, and r is an integer of from 1 to 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ—X—(CH₂)ₜ—, where s and t are independently integers of from 0 to 3, and X is —O—, —NR′—, —S—, —S(O)—, —S(O)₂—, or —S(O)₂NR′—. The substituent R′ in —NR′— and —S(O)₂NR′— is selected from hydrogen or unsubstituted C₁₋₆ alkyl.

As used herein, the term “heteroatom” is meant to include oxygen (O), nitrogen (N), sulfur (S) and silicon (Si).

The disclosure herein further relates to prodrugs and bioisosteres thereof. Suitable bioisosteres, for example, will include carboxylate replacements (phosphonic acids, phosphinic acids, sulfonic acids, sulfinic acids, and acidic heterocyclic groups such as tetrazoles). Suitable prodrugs will include those conventional groups known to hydrolyze and/or oxidize under physiological conditions to provide a compound of Formula I.

The terms “patient” and “subject” include primates (especially humans), domesticated companion animals (such as dogs, cats, horses, and the like) and livestock (such as cattle, pigs, sheep, and the like).

As used herein, the term “treating” or “treatment” encompasses both disease-modifying treatment and symptomatic treatment, either of which may be prophylactic (i.e., before the onset of symptoms, in order to prevent, delay or reduce the severity of symptoms) or therapeutic (i.e., after the onset of symptoms, in order to reduce the severity and/or duration of symptoms).

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. When a stereochemical depiction is shown, it is meant to refer to the compound in which one of the isomers is present and substantially free of the other isomer. ‘Substantially free of’ another isomer indicates at least an 80/20 ratio of the two isomers, more preferably 90/10, or 95/5 or more. In some embodiments, one of the isomers will be present in an amount of at least 99%.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are intended to be encompassed within the scope of the present disclosure. For example, the compounds may be prepared such that any number of hydrogen atoms are replaced with a deuterium (²H) isotope. The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere within this application. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics, which can contribute to enhanced safety, tolerability or efficacy during treatment. All isotopic variations of the compounds of the present disclosure, whether radioactive or not, are intended to be encompassed within the scope of the present disclosure.

### III. Embodiments of the Disclosure

### Methods of Treatment

In some aspects, provided herein are methods of treating a cancer comprising administering to a subject in need thereof an effective amount of a compound of Formula (I):

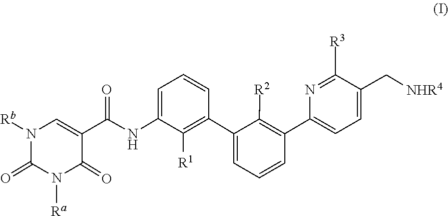

or a pharmaceutically acceptable salt thereof, wherein:
R¹ and R² are each independently selected from the group consisting of F, Cl, CH₃, and CF₃;
R³ is selected from the group consisting of F, Cl, CH₃, CF₃, —O—CH₃, and —O—CF₃;
R⁴ is selected from the group consisting of —Y and —X¹—Y wherein each X¹ is C₁₋₄ alkylene and, and Y is selected from the group consisting of C₃₋₆ cycloalkyl, C₄₋₆ heterocycloalkyl having 1 to 3 heteroatom ring vertices independely selected from the group consisting of N, O, and S, and 5- to 6-membered heteroaryl having 1 to 3 heteroatom ring vertices independently selected from the group consisting of N, O, and S, each of which is unsubstituted or substituted with one to two substituents independently selected from the group consisting of oxo, OH, C₁₋₄ alkyl, C₁₋₄ haloalkyl, C₁₋₄ hydroxyalkyl, C₁₋₄alkoxy, C₁₋₄haloalkoxy, and C₁₋₄ hydroxyalkoxy; and
R^{a} and R^{b} are independently selected from the group consisting of H, C₁₋₃ alkyl, and C₁₋₄ haloalkyl.

In some embodiments, R¹ is selected from the group consisting of Cl and CH₃. In some embodiments, R¹ is Cl. In some embodiments, R¹ is CH₃.

In some embodiments, R² is selected from the group consisting of Cl and CH₃. In some embodiments, R² is Cl. In some embodiments, R² is CH₃.

In some embodiments, R³ is selected from the group consisting of —O—CH₃ and —O—CF₃. In some embodiments, R³ is —O—CH₃. In some embodiments, R³ is —O—CF₃.

In some embodiments, R^{a} is selected from the group consisting of H, CH₃, and CF₃. In some embodiments, R^{a} is CH₃.

In some embodiments, R^{b} is selected from the group consisting of H, CH₃, and CF₃. In some embodiments, R^{b} is CH₃.

In some embodiments, the compound of Formula I has the Formula (Ia):

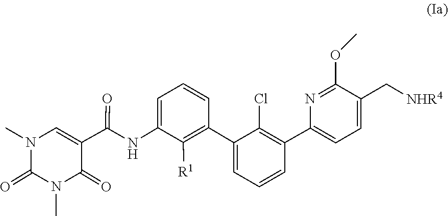

or a pharmaceutically acceptable salt thereof.

In some embodiments, —NH(R⁴) is selected from the group consisting of:

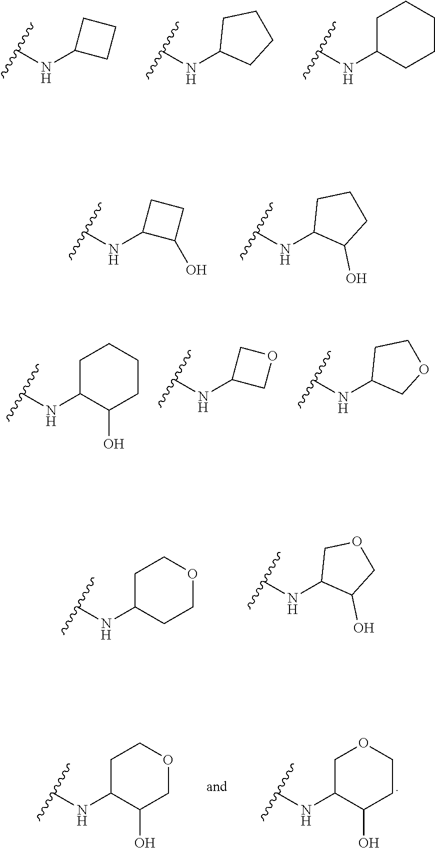

In some embodiments, —NH(R⁴) is selected from the group consisting of:

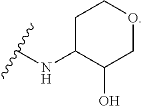 [SMILES: *NC1CCOCC1O]

In some embodiments, —NHR⁴ is selected from the group consisting of:

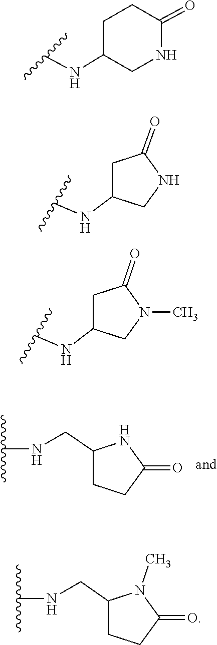

In some embodiments, —NHR⁴ is

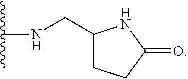 [SMILES: *NCC1CCC(=O)N1]

In some embodiments, the compound of Formula (I) is an optically pure or enriched isomer.

In some embodiments, the compound of Formula (I) is selected from a compound in Table 1.

As described herein, the disclosed methods for treating certain cancers do not require an extremely high concentration of a compound of Formula (I) in the blood. Instead, these compounds are sufficiently potent to provide a therapeutic benefit at lower blood plasma concentrations. Accordingly, in some embodiments, an effective amount of a compound of Formula (I) maintains a trough blood plasma concentration of no more than 1,000 ng/mL. In some embodiments, an effective amount of a compound of Formula (I) maintains a trough blood plasma concentration of no more than 750 ng/mL. In some embodiments, an effective amount of a compound of Formula (I) maintains a trough blood plasma concentration of no more than 500 ng/mL. In some embodiments, an effective amount of a compound of Formula (I) maintains a trough blood plasma concentration of no more than 400 ng/mL. In some embodiments, an effective amount of a compound of Formula (I) maintains a trough blood plasma concentration of no more than 300 ng/mL. In some embodiments, an effective amount of a compound of Formula (I) maintains a trough blood plasma concentration of no more than 200 ng/mL. In some embodiments, an effective amount of a compound of Formula (I) maintains a trough blood plasma concentration of no more than 100 ng/mL.

In some embodiments, the effective amount of a compound of Formula (I) maintains a trough blood plasma concentration from about 2 ng/mL to 1,000 ng/mL. In some embodiments, the effective amount of a compound of Formula (I) maintains a trough blood plasma concentration from about 5 ng/mL to 500 ng/mL. In some embodiments, the effective amount of a compound of Formula (I) maintains a trough blood plasma concentration from about 10 ng/mL to 400 ng/mL. In some embodiments, the effective amount of a compound of Formula (I) maintains a trough blood plasma concentration from about 20 ng/mL to 300 ng/mL. In some embodiments, the effective amount of a compound of Formula (I) maintains a trough blood plasma concentration from about 40 ng/mL to 200 ng/mL.

A number of cancers can be treated using the methods described herein. In some embodiments, the cancer is selected from the group consisting of melanoma, glioblastoma, esophagus tumor, nasopharyngeal carcinoma, uveal melanoma, lymphoma, lymphocytic lymphoma, primary CNS lymphoma, T-cell lymphoma, diffuse large B-cell lymphoma, primary mediastinal large B-cell lymphoma, prostate cancer, castration-resistant prostate cancer, chronic myelocytic leukemia, Kaposi's sarcoma fibrosarcoma, liposarcoma, chondrosarcoma, osteogenic sarcoma, angiosarcoma, lymphangiosarcoma, synovioma, meningioma, leiomyosarcoma, rhabdomyosarcoma, sarcoma of soft tissue, sarcoma, sepsis, biliary tumor, basal cell carcinoma, thymus neoplasm, cancer of the thyroid gland, cancer of the parathyroid gland, uterine cancer, cancer of the adrenal gland, liver infection, Merkel cell carcinoma, nerve tumor, follicle center lymphoma, colon cancer, Hodgkin's disease, non-Hodgkin's lymphoma, leukemia, chronic or acute leukemias including acute myeloid leukemia, chronic myeloid leukemia, acute lymphoblastic leukemia, chronic lymphocytic leukemia, multiple myeloma, ovary tumor, myelodysplastic syndrome, cutaneous or intraocular malignant melanoma, renal cell carcinoma, small-cell lung cancer, lung cancer, mesothelioma, liver cancer, breast cancer, squamous non-small cell lung cancer (SCLC), non-squamous NSCLC, colorectal cancer, ovarian cancer, gastric cancer, hepatocellular carcinoma, pancreatic carcinoma, pancreatic cancer, pancreatic ductal adenocarcinoma, squamous cell carcinoma of the head and neck, cancer of the head or neck, gastrointestinal tract, stomach cancer, HIV, hepatitis A, hepatitis B, hepatitis C, hepatitis D, herpes viruses, papillomaviruses, influenza, bone cancer, skin cancer, rectal cancer, cancer of the anal region, testicular cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the urethra, cancer of the penis, cancer of the bladder, cancer of the kidney, cancer of the ureter, carcinoma of the renal pelvis, neoplasm of the central nervous system (CNS), tumor angiogenesis, spinal axis tumor, brain stem glioma, pituitary adenoma, epidermoid cancer, asbestosis, carcinoma, adenocarcinoma, papillary carcinoma, cystadenocarcinoma, bronchogenic carcinoma, renal cell carcinoma, transitional cell carcinoma, choriocarcinoma, seminoma, embryonal carcinoma, wilm's tumor, pleomorphic adenoma, liver cell papilloma, renal tubular adenoma, cystadenoma, papilloma, adenoma, leiomyoma, rhabdomyoma, hemangioma, lymphangioma, osteoma, chondroma, lipoma and fibroma. In some embodiments each of the listed cancers are PD-L1 positive cancers.

In some embodiments, the cancer is colon cancer, renal cancer, colorectal cancer, gastric cancer, bladder cancer, melanoma, non-small cell lung cancer, Merkel cell carcinoma, liver cancer, breast cancer, and cancer of the head or neck. In some embodiments each of the listed cancers are PD-L1 positive cancers.

In some embodiments, the disease or disorder is colon cancer. In some embodiments, the cancer is renal cancer. In some embodiments, the cancer is colorectal cancer. In some embodiments, the cancer is gastric cancer. In some embodiments, the cancer is bladder cancer. In some embodiments, the cancer is melanoma. In some embodiments, the cancer is non-small cell lung cancer. In some embodiments, the cancer is liver cancer. In some embodiments, the cancer is breast cancer. In some embodiments each of the listed cancers are PD-L1 positive cancers.

In some embodiments, an effective amount of one or more additional therapeutic agents is further administered to the subject. In some embodiments, the one or more additional therapeutic agents is selected from the group consisting of a cytotoxic agent, a gene expression modulatory agent, a chemotherapeutic agent, an anti-cancer agent, an anti-angiogenic agent, an immunotherapeutic agent, an anti-hormonal agent, radiotherapy, a radiotherapeutic agent, an anti-neoplastic agent, and an anti-proliferation agent. In some embodiments, the one or more additional therapeutic agent is an antagonist of a chemokine and/or chemoattractant receptor, which includes but is not limited to, CCR1, CCR2, CCR3, CCR4, CCR5, CCR6, CCR7, CCR8, CCR9, CCR10, CCR11, CCR12, CXCR1, CXCR2, CXCR3, CXCR4, CXCR5, CXCR6, CXCR7, C3aR, and/or C5aR. Chemokine and/or chemoattractant receptor antagonists are known in the art and described in, for example, WO2007/002667, WO2007/002293, WO/2003/105853, WO/2007/022257, WO/2007/059108, WO/2007/044804, WO2007/115232, WO2007/115231, WO2008/147815, WO2010/030815, WO2010/075257, WO2011/163640, WO2010/054006, WO2010/051561, WO2011/035332, WO2013/082490, WO2013/082429, WO2014/085490, WO2014/100735, WO2014/089495, WO2015/084842, WO2016/187393, WO2017/127409, WO 2017/087607, WO2017/087610, WO2017/176620, WO2018/222598, WO2018/222601, WO2013/130811, WO2006/076644, WO2008/008431, WO2009/038847, WO2008/008375, WO2008/008374, WO2008/010934, WO2009/009740, WO2005/112925, WO2005/112916, WO2005/113513, WO2004/085384, WO2004/046092. Chemokine and/or chemoattractant receptor antagonists also include CCX354, CCX9588, CCX140, CCX872, CCX598, CCX6239, CCX9664, CCX2553, CCX3587, CCX3624, CCX 2991, CCX282, CCX025, CCX507, CCX430, CCX765, CCX224, CCX662, CCX650, CCX832, CCX168, CCX168-M1, CCX3022 and/or CCX3384.

Treatment methods provided herein include, in general, administration to a patient an effective amount of one or more compounds provided herein. Suitable patients include those patients suffering from or susceptible to (i.e., prophylactic treatment) a disorder or disease identified herein. Typical patients for treatment as described herein include mammals, particularly primates, especially humans. Other suitable patients include domesticated companion animals such as a dog, cat, horse, and the like, or a livestock animal such as cattle, pig, sheep and the like.

### Routes of Administration & Dosage

The routes of administration contemplated in the current disclosure include those known in the art for delivering an active agent for the treatment of a cancer. This includes, but is not limited to, oral administration, intratumor injection, intravenous administration, and subcutaneous injection. In some embodiments, the effective amount of a compound of Formula (I) is administered orally. In some embodiments, the effective amount of a compound of Formula (I) is administered via intratumor injection. In some embodiments, the effective amount of a compound of Formula (I) is administered intravenously. In some embodiments, the effective amount of a compound of Formula (I) is administered via subcutaneous injection.

In general, treatment methods provided herein comprise administering to a patient an effective amount of a compound of Formula (I) or one or more compounds provided herein. The effective amount may be an amount sufficient to modulate the PD-1/PD-L1 interaction, slow tumor growth, inhibit tumor growth, and/or reduce the tumor size in the subject. Preferably, the amount administered is sufficient to yield a plasma concentration of the compound (or its active metabolite, if the compound is a pro-drug) high enough to sufficiently modulate the PD-1/PD-L1 interaction. Treatment regimens may vary depending on the compound used and the particular condition to be treated; for treatment of most disorders, a frequency of administration of 4 times daily or less is preferred. In general, a dosage regimen of 2 times daily is more preferred, with once a day dosing particularly preferred. It will be understood, however, that the specific dose level and treatment regimen for any particular patient will depend upon a variety of factors including the activity of the specific compound employed, the age, body weight, general health, sex, diet, time of administration, route of administration, rate of excretion, drug combination (i.e., other drugs being administered to the patient) and the severity of the particular disease undergoing therapy, as well as the judgment of the prescribing medical practitioner. In general, the use of the minimum dose sufficient to provide effective therapy is preferred. Patients may generally be monitored for therapeutic effectiveness using medical or veterinary criteria suitable for the condition being treated or prevented.

Dosage levels of the order of from about 0.1 mg to about 140 mg per kilogram of body weight per day are useful in the treatment or preventions of conditions involving the PD-1/PD-L1 interaction (about 0.5 mg to about 7 g per human patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient.

For compounds administered orally, transdermally, intravenously, or subcutaneously, it is preferred that sufficient amount of the compound be administered to achieve a plasma concentration of 5 ng (nanograms)/mL-1 μg (micrograms)/mL plasma, more preferably sufficient compound to achieve a plasma concentration of 20 ng-0.5 μg/ml plasma should be administered, most preferably sufficient compound to achieve a plasma concentration of 30 ng/ml-200 ng/ml plasma should be administered.

Frequency of dosage may also vary depending on the compound used, the route of administration, and the particular disease treated. However, for treatment of most disorders, a dosage regimen of 4 times daily, three times daily, or less is preferred, with a dosage regimen of once daily or 2 times daily being particularly preferred. It will be understood, however, that the specific dose level for any particular patient will depend upon a variety of factors including the activity of the specific compound employed, the age, body weight, general health, sex, diet, time of administration, route of administration, and rate of excretion, drug combination (i.e., other drugs being administered to the patient), the severity of the particular disease undergoing therapy, and other factors, including the judgment of the prescribing medical practitioner.

### Pharmaceutical Compositions

Formula (I) when administered to a subject is typically in a pharmaceutical composition. The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form for oral administration and may be prepared by any of the methods well known in the art of pharmacy and drug delivery. All methods include the step of bringing the active ingredient into association with the carrier, which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition, the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, polyethylene glycol (PEG) of various average sizes (e.g., PEG400, PEG4000) and certain surfactants such as cremophor or solutol, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono- or di-glycerides, PEG esters and the like.

Aqueous suspensions contain the active materials in admixture with excipients suitable for the manufacture of aqueous suspensions. Such excipients are suspending agents, for example sodium carboxymethyl cellulose, methyl cellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, n-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The compounds of this disclosure may also be coupled with a carrier that is a suitable polymer for targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the disclosure may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like. In one embodiment of the disclosure, the compound of the disclosure is coupled to a polymer or semipermeable polymer matrix that is formed as a stent or stent-graft device.

In some embodiments, the pharmaceutical composition further comprises one or more additional therapeutic agents. In some embodiments, the one or more additional therapeutic agent is selected from the group consisting of an antimicrobial agent, an antiviral agent, a cytotoxic agent, a gene expression modulatory agent, a chemotherapeutic agent, an anti-cancer agent, an anti-angiogenic agent, an immunotherapeutic agent, an anti-hormonal agent, an anti-fibrotic agent, radiotherapy, a radiotherapeutic agent, an anti-neoplastic agent, and an anti-proliferation agent. In some embodiments, the one or more additional therapeutic agent is an antagonist of a chemokine and/or chemoattractant receptor, which includes but is not limited to, CCR1, CCR2, CCR3, CCR4, CCR5, CCR6, CCR7, CCR8, CCR9, CCR10, CCR11, CCR12, CXCR1, CXCR2, CXCR3, CXCR4, CXCR5, CXCR6, CXCR7, C3aR, and/or C5aR.

Chemokine and/or chemoattractant receptor antagonists are known in the art and described in, for example, WO2007/002667, WO2007/002293, WO/2003/105853, WO/2007/022257, WO/2007/059108, WO/2007/044804, WO2007/115232, WO2007/115231, WO2008/147815, WO2010/030815, WO2010/075257, WO2011/163640, WO2010/054006, WO2010/051561, WO2011/035332, WO2013/082490, WO2013/082429, WO2014/085490, WO2014/100735, WO2014/089495, WO2015/084842, WO2016/187393, WO2017/127409, WO 2017/087607, WO2017/087610, WO2017/176620, WO2018/222598, WO2018/222601, WO2013/130811, WO2006/076644, WO2008/008431, WO2009/038847, WO2008/008375, WO2008/008374, WO2008/010934, WO2009/009740, WO2005/112925, WO2005/112916, WO2005/113513, WO2004/085384, WO2004/046092. Chemokine and/or chemoattractant receptor antagonists also include CCX354, CCX9588, CCX140, CCX872, CCX598, CCX6239, CCX9664, CCX2553, CCX3587, CCX3624, CCX 2991, CCX282, CCX025, CCX507, CCX430, CCX765, CCX224, CCX662, CCX650, CCX832, CCX168, CCX168-M1, CCX3022 and/or CCX3384.

### EXAMPLES

The following Examples illustrate various methods of making compounds of this disclosure including compounds of Formulae (I) or (la). The following examples are offered to illustrate, but not to limit the claimed disclosure.

Reagents and solvents used below can be obtained from commercial sources such as Aldrich Chemical Co. (Milwaukee, Wis., USA). ¹H-NMR spectra were recorded on a Varian Mercury 400 MHz NMR spectrometer. Significant peaks are provided relative to TMS and are tabulated in the order: multiplicity (s, singlet; d, doublet; t, triplet; q, quartet; m, multiplet) and number of protons. Mass spectrometry results are reported as the ratio of mass over charge. In the examples, a single m/z value is reported for the M+H (or, as noted, M−H) ion containing the most common atomic isotopes. Isotope patterns correspond to the expected formula in all cases. Electrospray ionization (ESI) mass spectrometry analysis was conducted on a Hewlett-Packard MSD electrospray mass spectrometer using the HP 1100 HPLC for sample delivery. Normally the analyte was dissolved in methanol or CH₃CN at 0.1 mg/mL and 1 microliter was infused with the delivery solvent into the mass spectrometer, which scanned from 100 to 1000 Daltons. All compounds could be analyzed in the positive or negative ESI mode, using acetonitrile/water with 1% formic acid as the delivery solvent.

The following abbreviations are used in the Examples and throughout the description of the disclosure: TLC means Thin layer chromatography.

Compounds within the scope of this disclosure can be synthesized as described below, using a variety of reactions known to the skilled artisan. One skilled in the art will also recognize that alternative methods may be employed to synthesize the target compounds of this disclosure, and that the approaches described within the body of this document are not exhaustive, but do provide broadly applicable and practical routes to compounds of interest.

Certain molecules claimed in this patent can exist in different enantiomeric and diastereomeric forms and all such variants of these compounds are claimed unless a specific enantiomer is specified.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

### Example 1: N-(2′-chloro-3′-(5-((((3R,4R)-3-hydroxytetrahydro-2H-pyran-4-yl)amino)methyl)-6-methoxypyridin-2-yl)-2-methyl-[1,1′-biphenyl]-3-yl)-1,3-dimethyl-2,4-dioxo-1,2,3,4-tetrahydropyrimidine-5-carboxamide

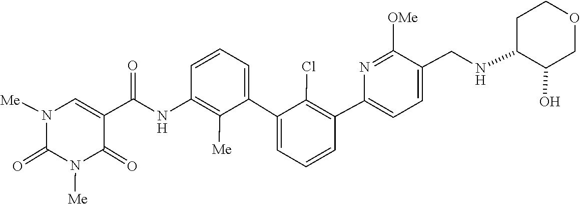 [SMILES: COc1nc(-c2cccc(-c3cccc(NC(=O)c4cn(C)c(=O)n(C)c4=O)c3C)c2Cl)ccc1CN[C@@H]1CCOC[C@@H]1O]

Step a: To a mixture of 1,3-dimethyl-N-(2-methyl-3-(4,4,5,5-tetramethyl-1,3,2-dioxaborolan-2-yl)phenyl)-2,4-dioxo-1,2,3,4-tetrahydropyrimidine-5-carboxamide (3.6 g, 9.0 mmol), 1,3-dibromo-2-chlorobenzene (6.9 g, 25.5 mmol), and K₂CO₃ (3.8 g, 27.5 mmol) in p-dioxane (40 mL) and DI H₂O (6 mL) was added Pd(dppf)Cl₂ complex with dichloromethane (912 mg, 1.12 mmol). The reaction mixture was degassed (N₂) for 2 min and stirred under N₂ at 90° C. for 2 h. The reaction mixture was diluted with EtOAc, filtered through Celite, washed with brine and dried over MgSO₄. The solvent was removed under reduced pressure and the residue was purified by silica gel flash chromatography (5 to 100% EtOAc in hexanes followed by 0 to 5% MeOH in EtOAc) to give N-(3′-bromo-2′-chloro-2-methyl-[1,1′-biphenyl]-3-yl)-1,3-dimethyl-2,4-dioxo-1,2,3,4-tetrahydropyrimidine-5-carboxamide. MS: (ES) m/z calculated for C₂₀H₁₈BrClN₃O₃ [M+H]⁺ 462.0, found 462.0.

Step b: To a mixture of N-(3′-bromo-2′-chloro-2-methyl-[1,1′-biphenyl]-3-yl)-1,3-dimethyl-2,4-dioxo-1,2,3,4-tetrahydropyrimidine-5-carboxamide (1.4 g, 3.03 mmol), pinacol diborane (1.0 g, 3.94 mmol), and KOAc (1.2 g, 10.2 mmol) in p-dioxane (18 mL) was added Pd(dppf)Cl₂ complex with dichloromethane (350 mg, 0.43 mmol). The reaction mixture was degassed (N₂) for 2 min and stirred under N₂ at 90° C. for 3 h. The reaction mixture was diluted with EtOAc, filtered through Celite, washed with brine and dried over MgSO₄. The solvent was removed under reduced pressure and the residue was purified by silica gel flash chromatography (10 to 60% EtOAc in hexanes) to give N-(2′-chloro-2-methyl-3′-(4,4,5,5-tetramethyl-1,3,2-dioxaborolan-2-yl)-[1,1′-biphenyl]-3-yl)-1,3-dimethyl-2,4-dioxo-1,2,3,4-tetrahydropyrimidine-5-carboxamide. MS: (ES) m/z calculated for C₂₆H₃₀BClN₃O₅ [M+H]⁺ 510.2, found 510.1.

Step c: To a mixture of N-(2′-chloro-2-methyl-3′-(4,4,5,5-tetramethyl-1,3,2-dioxaborolan-2-yl)-[1,1′-biphenyl]-3-yl)-1,3-dimethyl-2,4-dioxo-1,2,3,4-tetrahydropyrimidine-5-carboxamide (400 mg, 0.78 mmol), 6-chloro-2-methoxynicotinaldehyde (200 mg, 1.17 mmol), and K₂CO₃ (350 mg, 2.53 mmol) in p-dioxane (10 mL) and DI H₂O (2 mL) was added Pd(dppf)Cl₂ complex with dichloromethane (70 mg, 0.086 mmol). The reaction mixture was degassed (N₂) for 2 min and stirred under N₂ at 95° C. for 2 h. The reaction mixture was diluted with EtOAc, filtered through Celite, washed with brine and dried over MgSO₄. The solvent was removed under reduced pressure and the residue was purified by silica gel flash chromatography (10 to 65% EtOAc in hexanes) to give N-(2′-chloro-3′-(5-formyl-6-methoxypyridin-2-yl)-2-methyl-[1,1′-biphenyl]-3-yl)-1,3-dimethyl-2,4-dioxo-1,2,3,4-tetrahydropyrimidine-5-carboxamide. MS: (ES) m/z calculated for C₂₇H₂₄ClN₄O₅ [M+H]⁺ 519.1, found 519.1.

Step d: To a stirred solution of N-(2′-chloro-3′-(5-formyl-6-methoxypyridin-2-yl)-2-methyl-[1,1′-biphenyl]-3-yl)-1,3-dimethyl-2,4-dioxo-1,2,3,4-tetrahydropyrimidine-5-carboxamide (40 mg, 0.077 mmol) and (3R,4R)-4-aminotetrahydro-2H-pyran-3-ol hydrochloride (24 mg, 0.154 mmol) in dichloroethane (2 mL) and ethanol (1 mL) was added triethylamine (2 drops) then followed by acetic acid (2 drops). The reaction mixture was stirred at 70° C. for 1 hr. The mixture was then cooled to 0° C. and NaCNBH₃ (10 mg, 0.154 mmol) was added slowly. The mixture was stirred at 0° C. for 10 minutes. The mixture was passed through syringe filter and then purified by preparative HPLC (0 to 40% to 100% Acetonitrile/H₂O) to give N-(2′-chloro-3′-(5-((((3R,4R)-3-hydroxytetrahydro-2H-pyran-4-yl)amino)methyl)-6-methoxypyridin-2-yl)-2-methyl-[1,1′-biphenyl]-3-yl)-1,3-dimethyl-2,4-dioxo-1,2,3,4-tetrahydropyrimidine-5-carboxamide. ¹H NMR (400 MHz, CD₃OD) δ 11.18 (s, 1H), 8.63 (s, 1H), 8.13-8.06 (m, 1H), 7.88 (d, J=7.6 Hz, 1H), 7.61 (dd, J=7.6, 1.7 Hz, 1H), 7.49 (t, J=7.6 Hz, 1H), 7.39-7.25 (m, 3H), 7.00 (d, J=1.1 Hz, 1H), 4.35 (d, J=13.3 Hz, 1H), 4.24 (d, J=13.2 Hz, 1H), 4.11-3.93 (m, 6H), 3.61-3.36 (m, 10H), 2.13 (s, 4H), 1.87 (d, J=12.4 Hz, 1H). MS: (ES) m/z calculated for C₃₂H₃₄ClN₅O₆ [M+H]⁺ 620.2, found 620.2.

### Example 2: (S)—N-(2′-chloro-3′-(6-methoxy-5-((((5-oxopyrrolidin-2-yl)methyl)amino)methyl)pyridin-2-yl)-2-methyl-[1,1′-biphenyl]-3-yl)-1,3-dimethyl-2,4-dioxo-1,2,3,4-tetrahydropyrimidine-5-carboxamide

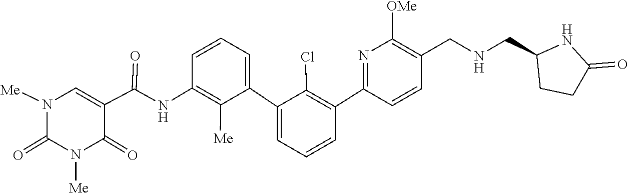 [SMILES: COc1nc(-c2cccc(-c3cccc(NC(=O)c4cn(C)c(=O)n(C)c4=O)c3C)c2Cl)ccc1CNC[C@@H]1CCC(=O)N1]

The title compound was prepared from N-(2′-chloro-3′-(5-formyl-6-methoxypyridin-2-yl)-2-methyl-[1,1′-biphenyl]-3-yl)-1,3-dimethyl-2,4-dioxo-1,2,3,4-tetrahydropyrimidine-5-carboxamide and (S)-5-aminomethylpyrrolidin-2-one hydrochloride using the same procedure as in Example 1. The crude product was purified by reverse phase HPLC (C18 column, acetonitrile/H₂O with 0.1% TFA as eluent) to give the desired product (S)—N-(2′-chloro-3′-(6-methoxy-5-((((5-oxopyrrolidin-2-yl)methyl)amino)methyl)pyridin-2-yl)-2-methyl-[1,1′-biphenyl]-3-yl)-1,3-dimethyl-2,4-dioxo-1,2,3,4-tetrahydropyrimidine-5-carboxamide. ¹H NMR (400 MHz, CD₃OD) δ 8.63 (d, J=1.1 Hz, 1H), 8.12 (dd, J=1.9, 1.3 Hz, 1H), 7.88 (d, J=7.6 Hz, 1H), 7.61 (d, J=7.7 Hz, 1H), 7.50 (t, J=7.6 Hz, 1H), 7.41-7.25 (m, 3H), 7.00 (d, J=7.5 Hz, 1H), 4.34 (d, J=2.0 Hz, 2H), 4.13-4.00 (m, 4H), 3.55 (d, J=1.0 Hz, 3H), 3.39 (d, J=1.0 Hz, 3H), 3.34-3.22 (m, 2H), 2.49-2.32 (m, 3H), 2.13 (s, 3H), 1.92 (q, J=7.5 Hz, 1H). MS: (ES) m/z calculated for C₃₂H₃₃ClN₆O₅ [M+H]⁺ 617.2, found 617.2.

### Example 3: (S)—N′-(2,2′-dichloro-3′-(6-methoxy-5-((((5-oxopyrrolidin-2-yl)methyl)amino)methyl)pyridin-2-yl)-[1,1′-biphenyl]-3-yl)-1,3-dimethyl-2,4-dioxo-1,2,3,4-tetrahydropyrimidine-5-carboxamide

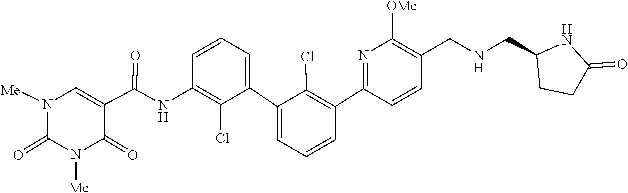 [SMILES: COc1nc(-c2cccc(-c3cccc(NC(=O)c4cn(C)c(=O)n(C)c4=O)c3Cl)c2Cl)ccc1CNC[C@@H]1CCC(=O)N1]

The title compound was prepared from N-(2,2′-dichloro-3′-(5-formyl-6-methoxypyridin-2-yl)-[1,1′-biphenyl]-3-yl)-1,3-dimethyl-2,4-dioxo-1,2,3,4-tetrahydropyrimidine-5-carboxamide and (A)-5-(aminomethyl)pyrrolidin-2-one hydrochloride using a procedure similar to Example 1. The crude product was purified by preparative HPLC (C18 column, MeCN/H₂O with 0.1% TFA as eluent) to give (S)—N-(2,2′-dichloro-3′-(6-methoxy-5-((((5-oxopyrrolidin-2-yl)methyl)amino)methyl)pyridin-2-yl)-[1,1′-biphenyl]-3-yl)-1,3-dimethyl-2,4-dioxo-1,2,3,4-tetrahydropyrimidine-5-carboxamide. ¹H NMR (400 MHz, CD₃OD) δ 11.69 (s, 1H), 8.66 (s, 1H), 8.54 (d, J=8.3 Hz, 1H), 7.93-7.85 (m, 1H), 7.65 (dd, J=7.8, 1.8 Hz, 1H), 7.51 (dd, J=7.7, 7.7 Hz, 1H), 7.45-7.35 (m, 3H), 7.10 (d, J=7.6 Hz, 1H), 4.34 (s, 2H), 4.14-4.01 (m, 4H), 3.56 (d, J=1.6 Hz, 3H), 3.39 (d, J=1.8 Hz, 3H), 3.30-3.20 (m, 3H), 2.40 (dd, J=11.8, 11.1 Hz, 2H), 2.03 (d, J=1.7 Hz, 1H), 1.92 (d, J=6.9 Hz, 1H). MS: (ES) m/z calculated for C₃₁H₃₁Cl₂N₆O₅ [M+H]⁺ 637.2, found 637.2.

### Biological Example 1: Enzyme-Linked Immunosorbent Assay—ELISA

96 Well plates were coated with 1 μg/mL of human PD-L1 (obtained from R&D) in PBS overnight at 4° C. The wells were then blocked with 2% BSA in PBS (W/V) with 0.05% TWEEN-20 for 1 hour at 37° C. The plates were washed 3 times with PBS/0.05% TWEEN-20 and the compounds were serial diluted (1:5) in dilution medium and added to the ELISA plates. Human PD-1 and biotin 0.3 μg/mL (ACRO Biosystems) were added and incubated for 1 hour at 37° C. then washed 3 times with PBS/0.05% TWEEN-20. A second block was performed with 2% BSA in PBS (W/V)/0.05% TWEEN-20 for 10 min at 37° C. and the plates were washed 3 times with PBS/0.05% TWEEN-20. Streptavidin-HRP was added for 1 hour at 37° C. then the plates were washed 3 times with PBS/0.05% TWEEN-20. TMB substrate was added and reacted for 20 min at 37° C. A stop solution (2 N aqueous H₂SO₄) was added. The absorbance was read at 450 nm using a micro-plate spectrophotometer. The results are shown in Table 1: IC₅₀ values are provided as follows: from 1000 to 10,000 nM (+); from 10 up to 1000 nM (++); less than 10 nM (+++).

TABLE 1

| | | ELISA |
|---|---|---|
| Compound | Structure | IC₅₀ |
| 2.001 | 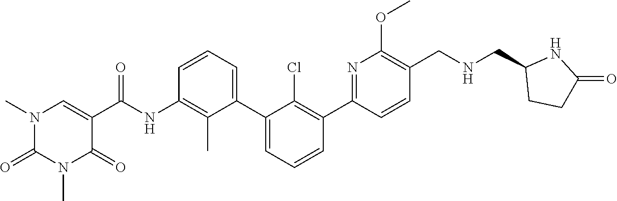 [SMILES: COc1nc(-c2cccc(-c3cccc(NC(=O)c4cn(C)c(=O)n(C)c4=O)c3C)c2Cl)ccc1CNC[C@@H]1CCC(=O)N1] | +++ |
| 2.002 | 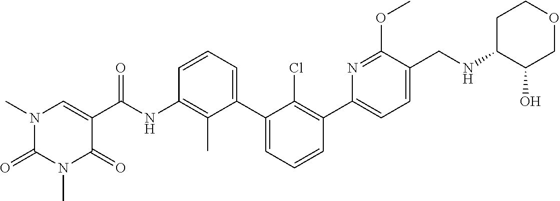 [SMILES: COc1nc(-c2cccc(-c3cccc(NC(=O)c4cn(C)c(=O)n(C)c4=O)c3C)c2Cl)ccc1CN[C@@H]1CCOC[C@@H]1O] | +++ |
| 2.003 | 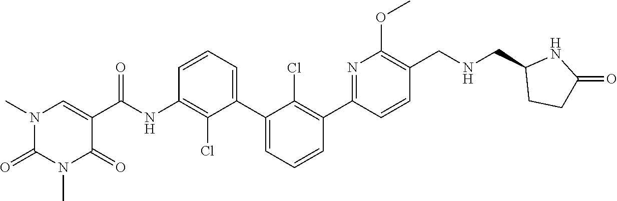 [SMILES: COc1nc(-c2cccc(-c3cccc(NC(=O)c4cn(C)c(=O)n(C)c4=O)c3Cl)c2Cl)ccc1CNC[C@@H]1CCC(=O)N1] | +++ |

### Biological Example 2: Anti-Tumor Effect of Compounds 2.001, 2.002, and 2.003

This example demonstrates the biologic anti-tumor effects of Compound 2.001, 2.002, and 2.003 disclosed herein.

ELISA: This assay was performed substantially as described in Biological Example 1.

Cell lines and cell culture: CHO cells, constitutively expressing the TCR agonist and PD-L1 were grown with Ham's solution supplemented with 10% FBS and used for cell-based assay. T lymphocyte-like cell line (Jurkat) modified to constitutively express PD-1 and carrying a luciferase reporter gene driven by TCR-inducible NFAT response element (Effector Cells, ECs) (Jurkat PD-1) were grown in RPMI supplemented with 10% FBS and IX penicillin-streptomycin and used for cell-based assay. Human melanoma cell line A375 and human breast cancer cell line MDA-MB-231 were obtained from ATTC and grown in DMEM supplemented with 10% FBS and IX penicillin-streptomycin. Human PBMCs were isolated in-house and grown in RPMI supplemented with 10% FBS and IX penicillin-streptomycin.

PD-1/PD-L1 Blockade Cell-based Assay; 6×10⁴ Cho PD-L1 cells were seeded in 96 well plates overnight at 37° C. After washing the cells with PBS IX, 40 μl of compound diluted with 1% FBS RPMI (starting concentration of 5 μM followed by 1:5 dilution) along with 40 μl of TJurkat PD-1 (1×10⁶ cell/ml) were added to each well and incubated for 6 hours at 37° C. After cooling the cells to room temperature, 80 μl of Bio-Glo Reagent (Promega, Madison, Wis.) was added to the media and relative light units (RLU) were measured on a FlexStation 3 plate reader at a speed of 500 ms/well. When the two cells were co-culture together, the PD-1/PD-L1 interaction inhibits TCR signaling and NFAT-RE-mediated luminescence. By adding and anti-PD-1 or anti-PD-L1 antibodies/compounds that blocks the PD-1/PD-L1 interaction releases the inhibition signal and results in TCR activation and NFAT-RE-mediated luminescence.

PBMCs Isolation: Peripheral blood mononuclear cells (PBMCs) were isolated from buffy coat from LRS chambers (leukoreduction systems) from healthy donors by density gradient centrifugation using StemCell SepMate™-50 tubes (STEMCELL Technologies, Vancouver, Calif.) containing Ficoll-Paque Plus (Sigma Aldrich Inc., St. Louis, Mo.).

Generation of monocyte derived dendritic cells: CD14⁺ monocytes were isolated from PBMCs by magnetic separation using human CD14⁺ MicroBeads (MACS Miltenyi Biotech, Bergisch Gladbach, Germany) and an autoMACS® Pro Separator. Isolated monocytes were plated at a concentration of 1×10⁶ cells/ml and differentiated to dendritic cells by adding GM-CSF (100 ng/ml) and IL-4 (50 ng/ml) for 6 days. Fresh media with cytokine supplement were added on day 0 and day 2. Mature dendritic cells were induced on day 6 by addition of IL-6 (2000 IU/ml), IL-1B (400 IU/ml) (Peprotech, Inc. Rocky Hill, N.J.), TNFalpha (2000 IU/ml) and PGE2 (2 ug/ml) (Sigma Aldrich, Inc.) and cultured for 24 hours.

Preparation of human effector cells: CD4⁺ T-Cells were isolated from PBMCs by magnetic separation using human CD4⁺ MicroBeads (MACS Miltenyi Biotech) and an autoMACS® Pro Separator.

Mixed Lymphocyte Reaction (MLR): DC and CD4⁺T-Cells from unmatched donors were cultured together in a ratio of 1:10 for 5 days in 96 well-flat bottom plates (Thermo Scientific). Test compounds were added as indicated at starting concentration of 1 μM with 1:4 dilutions with DMSO. PD-L1 antibody (AZ Medi4736 analog) and isotype control (Human IgG1, Kappa Isotype Control) (CrownBio, Beijing) were used as positive and negative control respectively. Supernatants were harvested after 5 days of incubation and detection of human IFNg was performed by ELISA using Human IFN-gamma DuoSet ELISA (R&D Systems, Minneapolis) as per manufacturer's instructions.

In Vitro Immunotherapy Potency Assay; A375-eGFP-Puro Cells (ATCC) were grown in complete medium (DMEM+10% FBS+P/S 1×) containing 1 ug/ml of puromycin. Human peripheral blood mononuclear cells (hPBMCs) were isolated from huffy coat from LRS chambers (leukoreduction systems) from healthy donors by density gradient centrifugation using StemCell SepMate™-50 tubes (STEMCELL Technologies, Vancouver, Calif.) containing Ficoll-Paque Plus (Sigma Aldrich Inc., St. Louis, Mo.). Freshly Isolated hPBMCs were stimulated with 100 ng/ml of Staphylococcal Enterotoxin B (SEB) (EMD Milipore, Cat 324798) for three days. Cells were washed twice and re-suspended into regular growth media. 3×10⁴ A375-eGFP-Puro cells were seeded in 96-well clear bottom black TC treated plates in a final volume of 100 ul (Corning). Test compounds or anti-human PD-L1 antibody (AZ Medi4736 analog, CrownBio, Beijing) were added to the wells at different concentrations. SEB stimulated hPBMCs were added to the wells at a ratio of E:T (Effector cell: Target Cell) of 2:1. Mixed cells were incubated for 96 to 120 hours at 37° C. in 5% CO₂. Medium was carefully aspired and 100 μl of PBS 1× was added to each well. Fluorescence from A375-eGFP cells was detected using FlexStation 3 plate reader.

Dimerization Assay PD-L1 protein dimerization was assessed in vitro by chemiluminescent detection using PathHunter® Dimerization assay (DiscoverX, Fremont, Calif.). The Assay was performed following vendor's protocol. 2×10⁴ U2OS cells were plated in 96-well white bottom TC treated plates (Costar, San Jose, Calif.) in a final volume of 100 ul. ChemoCentryx compounds or anti-human PD-L1 antibody (AZ Medi4736 analog, CrownBio, Beijing) were added to experimental cells at different concentrations and incubated for 16 hours at 37° C. in 5% CO₂. 110 ul of PathHunter Flash detection reagent (DiscoverX) was added to each well and incubated for 1 hour at room temperature in the dark. Chemiluminescent signal was measured on a FlexStation 3 plate reader (Molecular Devices, San Jose, Calif.) at a speed of 100 ms/well.

Internalization Assay. MC38-hPD-L1 cells (GenOway S.A., France) and RKO cells (ATCC) growing at 37° C. in 5% CO₂ were detached, resuspended in cold FACS buffer (PBS IX with 10% FBS and 0.1% azide) and added to 96-well assay plates (V-Bottom) (Axygen, Union City, Calif.) at a concentration of 10×10⁴ cells/well. ChemoCentryx compounds or anti-human PD-L1 antibody (AZ Medi4736 analog) were added to the wells at different concentrations and incubated for 2 hours at 37° C. or 4° C. Cells were washed twice with iced cold FACS buffer and stained with recombinant rabbit monoclonal anti human PD-L1 antibody ([28-8] (PE) (ab209962), Abeam) or recombinant rabbit monoclonal IgG Isotype control ([EPR25A] (PE) (ab209478), Abeam) for 30 minutes on ice. Cells were washed twice with FACS buffer before performing FACS analysis. Data were analyzed using FlowJo software.

Generation and culture of MC38-hPD-L1 cells; The compounds are only known to cross-react with human PD-L1, therefore a syngeneic tumor model with murine MC-38 colon tumor cells expressing human PD-L1 (MC38-hPD-L1 tumor model) was used. The MC38-hPD-L1 cells were generated by GenOway. Endogenous mouse PD-L1 was first knocked out in MC38 cells using CRISPR technology, then human PDL1 was stably transfected in these mouse PD-L1 Knock-out MC38 cells. The MC38-hPD-L1 cells were cultured under standard conditions for MC38 cells (DMEM with 10% fetal bovine serum and Penicillin/Streptomycin) with G418 for maintaining transgene expression. 2 days prior to inoculation of these cells to mice, cells were trypsinized and seeded without antibiotics.

In Vivo Studies; 8-week-old, female C57BL/6 mice were injected s.c. with 5×10⁵ MC38-hPD-L1 cells in the right flank. 9 days after tumor inoculation mice were randomly assigned to treatment groups based on tumor size. Only mice that developed measurable tumors were enrolled in the studies. Anti-PD-L1 (Durvalumab) or isotype control was given i.p. twice a week at 100 ug per mouse per dose for 2 weeks. Compound 2.001 and Compound 2.002 suspended in 1% HPMC was dosed p.o. daily at indicated doses, dosing volume 100 μl per mouse. The vehicle, 1% HPMC, was dosed at the same volume with the same frequency for control animals.

Tumor volume was measured three times per week using a digital caliper and calculated as (width²*length/2). Mice were sacrificed when tumor volumes reached 2,000 mm³ in accordance with IACUC guidelines.

Tumor width (W) and length (L) were measured with calipers 3 times a week and the tumor volume was calculated using the formula V=(W(2)×L)/2. When tumors reach 2000 mm³ mice were sacrificed and tumor excised for further analysis.

Cellular phenotyping of tumor infiltrates; Excised tumors were finely chopped with a blade and meshed through 200 um sieve. Cells were then filtered through a 70 μM sieve. Cells were washed and resuspended in FACS buffer (PBS IX with 10% FBS and 0.1% azide).

Antibodies for flow cytometry were obtained from BioLegend (San Diego, Calif.). Flow cytometer panel included CD45 in FITC, PD-L1 in PE, CD8 in APC, CD4 in APC-Cy7. Flow cytometry data were acquired with a FACSCanto II (BD Biosciences, San Jose, Calif.) cytometer and analyzed using FlowJO vl0.2 (FlowJo, Ashland, Oreg.).

### Results:

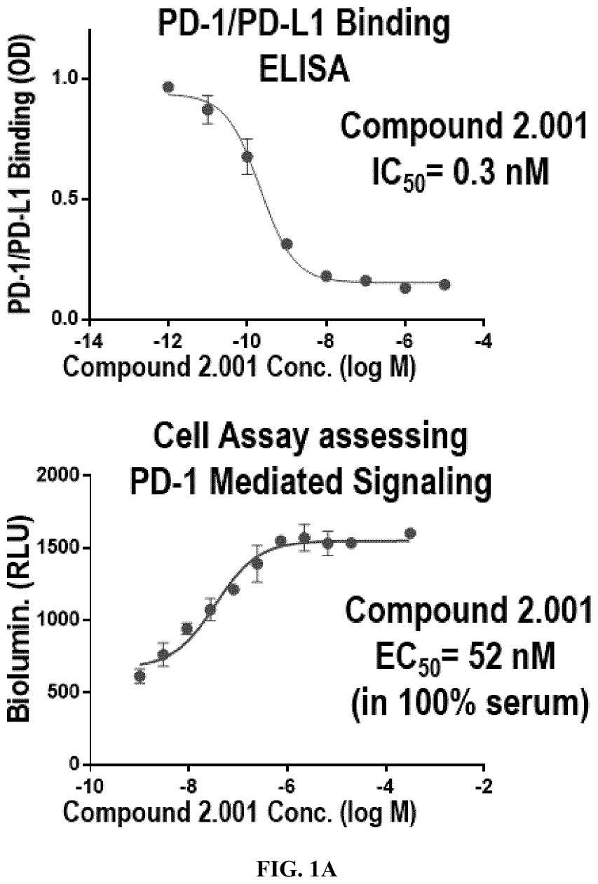

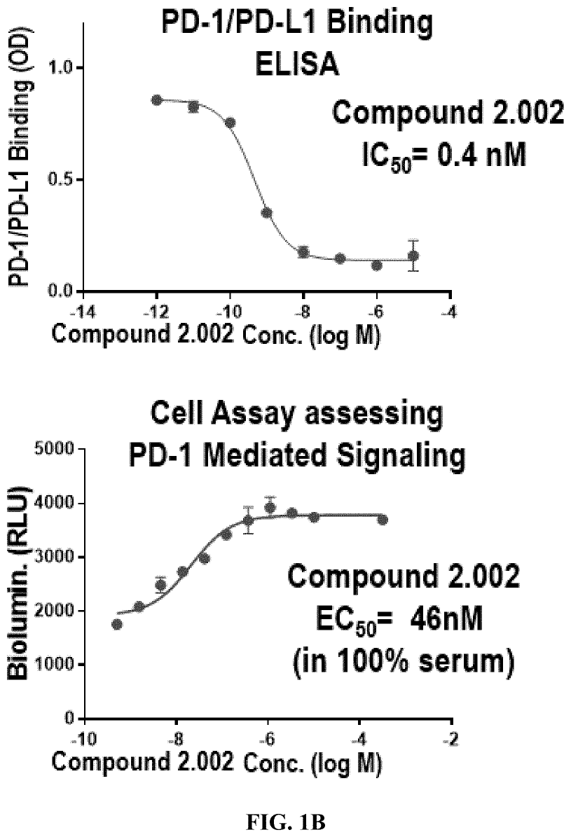

In an enzyme-linked immunosorbent assay (ELISA), Compound 2.001 and 2.002 both potently inhibited direct interaction of PD-L1 to PD-1. The average IC₅₀ of 2.001 and 2.002 from multiple assays are 0.3 nM and 0.4 nM, respectively (FIG. 1). In a cell based assay assessing PD-1 mediated downstream signaling, these compounds enhances the NFAT promoter-driven luciferase expression that is suppressed by PD-L1/PD-1 interaction. The average EC₅₀ of Compound 2.001 and 2.002 in this assay are 52 nM and 46 nM, respectively.

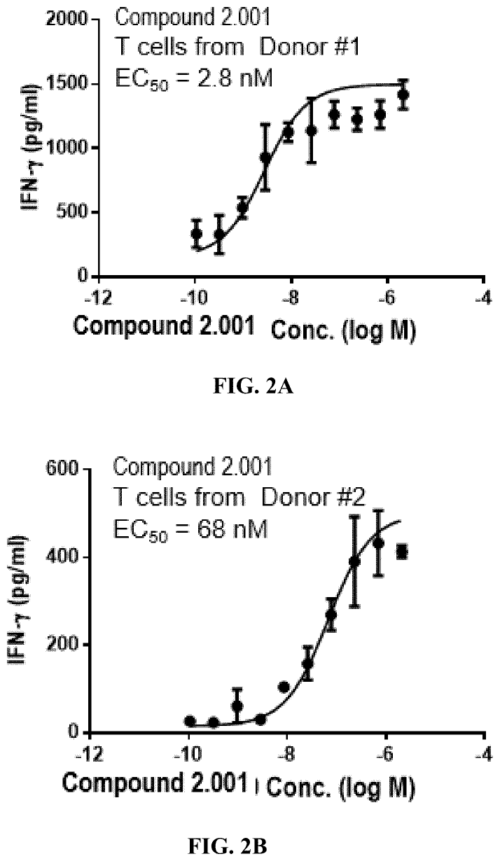

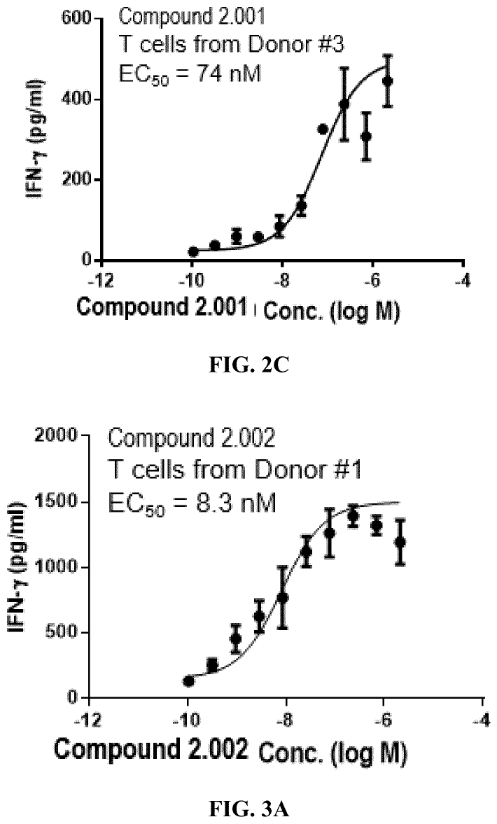

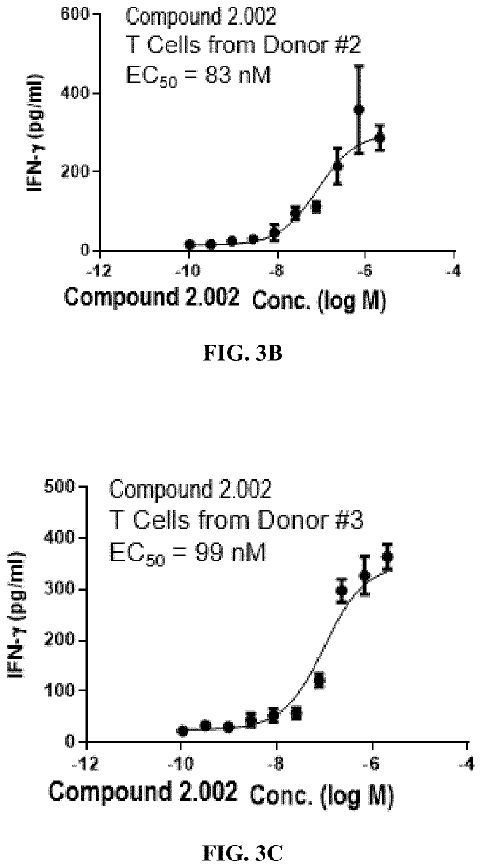

In the mixed lymphocyte reaction (MLR) assay (FIG. 2 and FIG. 3), Compound 2.001 and Compound 2.002 dose dependently increase the release of INFgamma from human T cells. The responsiveness of T cells from different donors varies, but both compounds exhibit EC₅₀ less than 100 nM with different T cells.

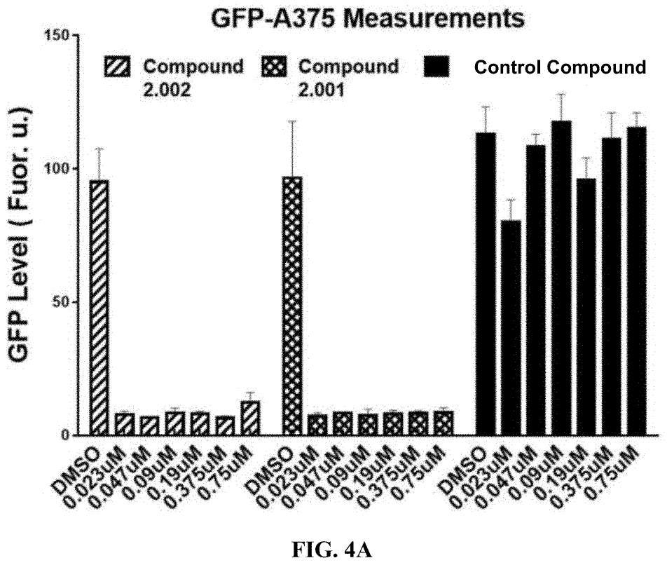

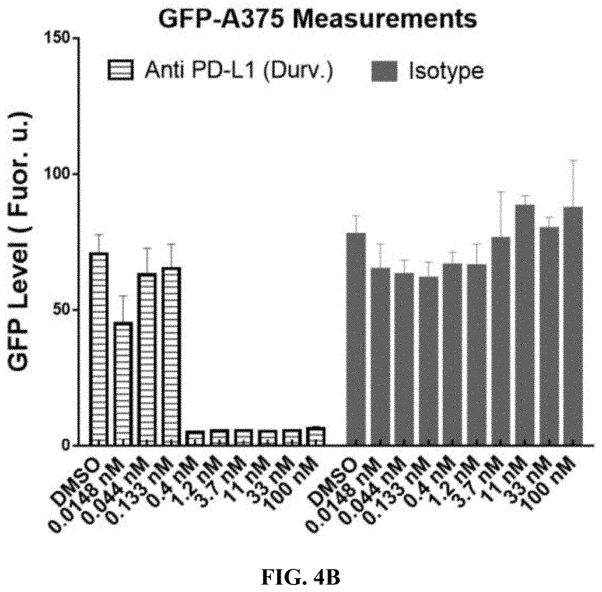

In the presence of pre-stimulated primary human PBMCs, Compound 2.001 and 2.002 promote killing of the GFP labeled human cancer cell line A375 (FIG. 4A). Durvalumab, the FDA-approved anti-PD-L1 antibody, was used as a positive control and a comparator in this study (FIG. 4B).

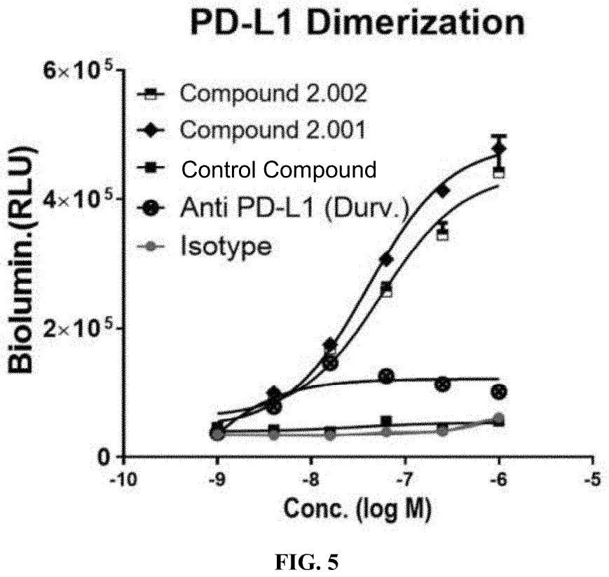

In the pathhunter assay (dimerization assay), dimerization of 2 PD-L1 molecules would bring the 2 enzyme subunits together and form a functional enzyme, which generates bioluminescent signal. Compound 2.001 and 2.002 both strongly induced the dimerization signal, while a control compound and the anti-PD-L1 antibody, do not induce such signal (FIG. 5).

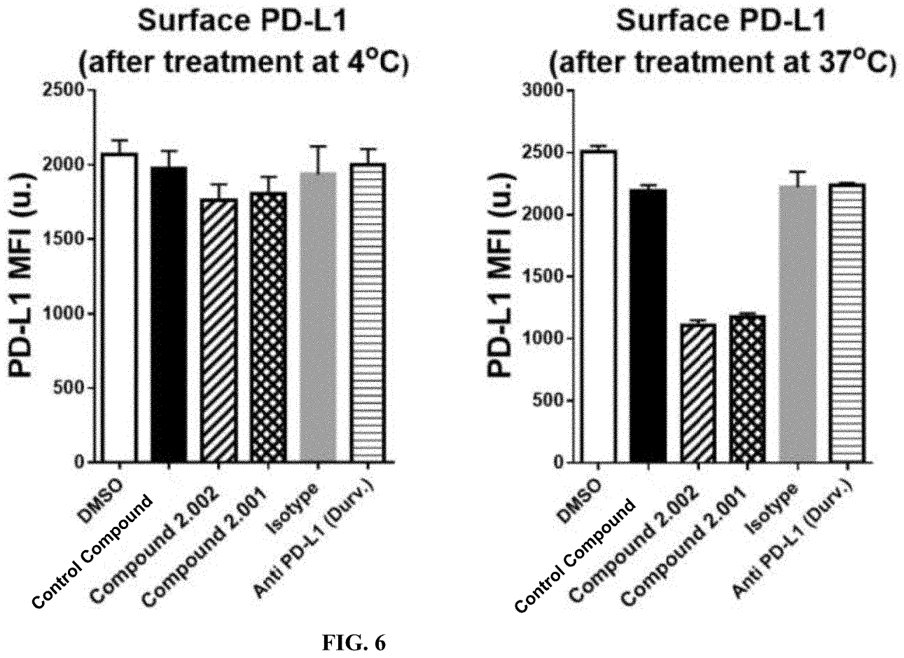

Surface PD-L1 on a tumor cell line was measured by flow cytometry. The binding of the detection antibody to PD-L1 is not affected by small molecule inhibitors, as illustrated by minimal changes of PD-L1 staining with compound treatment at 4° C. At 37° C., the temperature that allows receptor internalization, Compound 2.001 and 2.002 profoundly reduces surface PD-L1 levels on cell surface (FIG. 6). Anti-PD-L1 antibody has no effect on PD-L1 surface levels. These facts suggest Compound 2.001 and 2.002 promotes PD-L1 internalization.

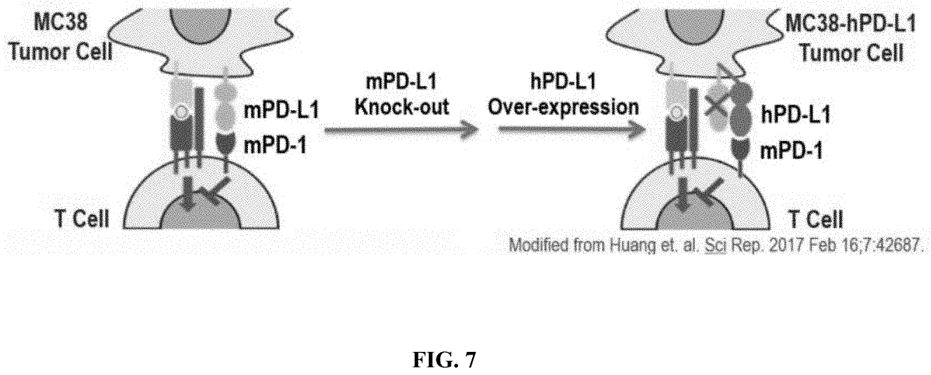

A mouse tumor cell line, MC38, in which the mouse PD-L1 was replaced with a human PD-L1 transgene, was used to induce tumor growth in mouse (FIG. 7). We confirmed that human and mouse PD-L1 binds to mouse PD-1 with similar affinity, and our PD-L1 inhibitors block human PD-L1 interaction with mouse PD-1 with similar potency (data not shown).

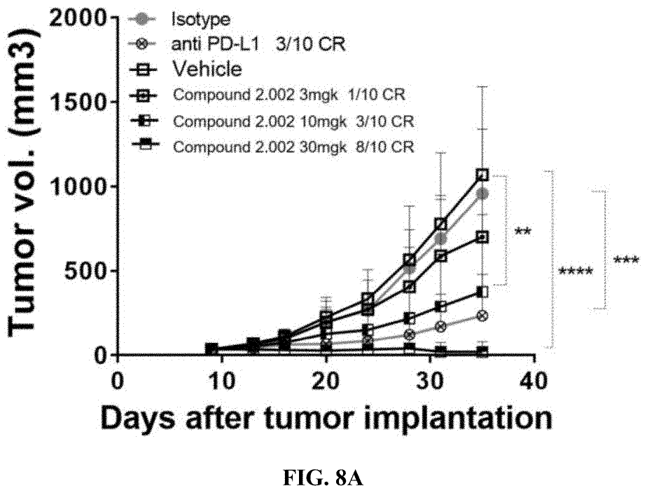

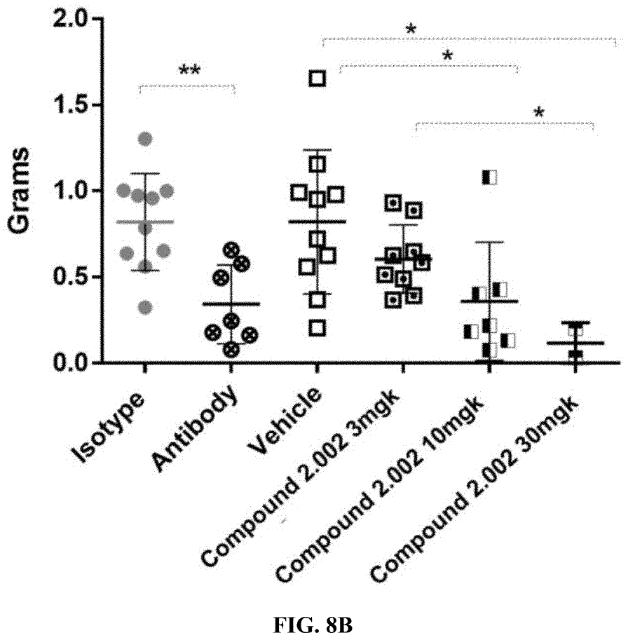

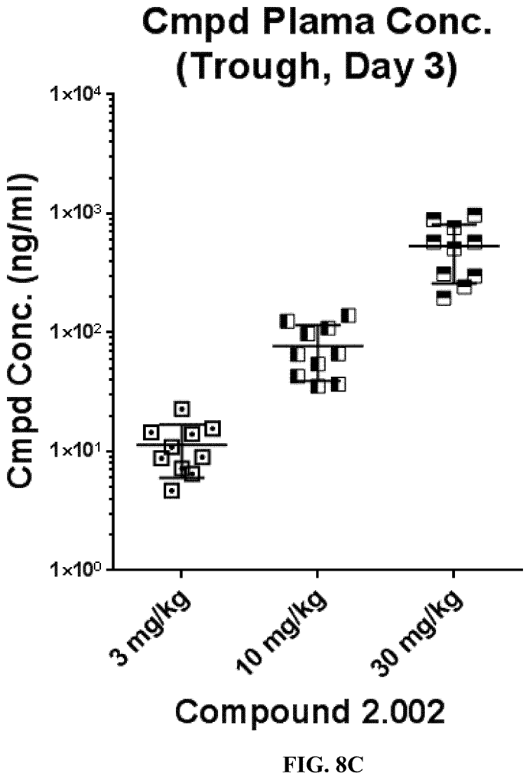

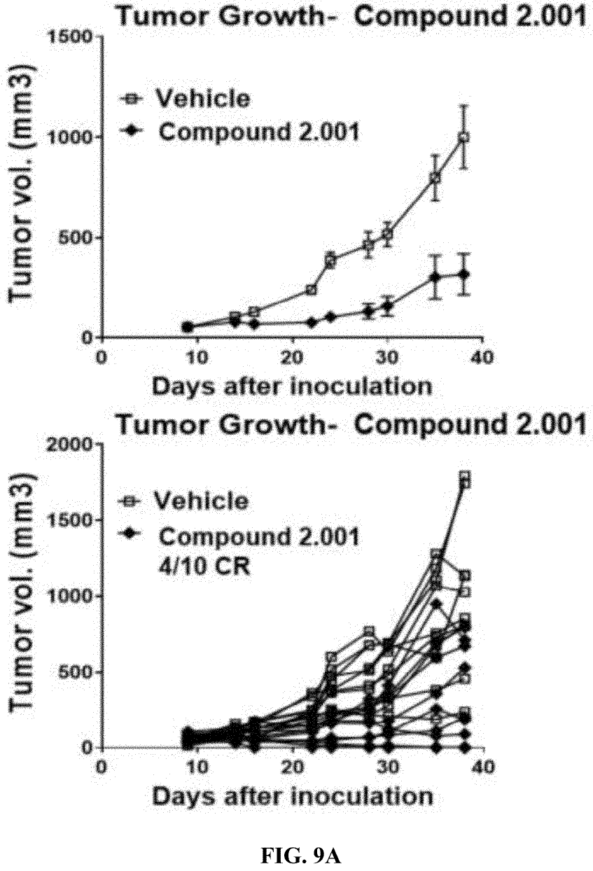

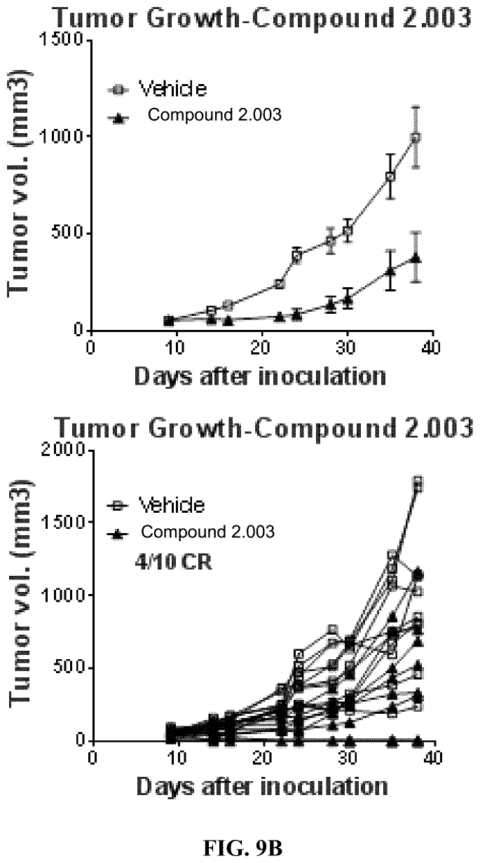

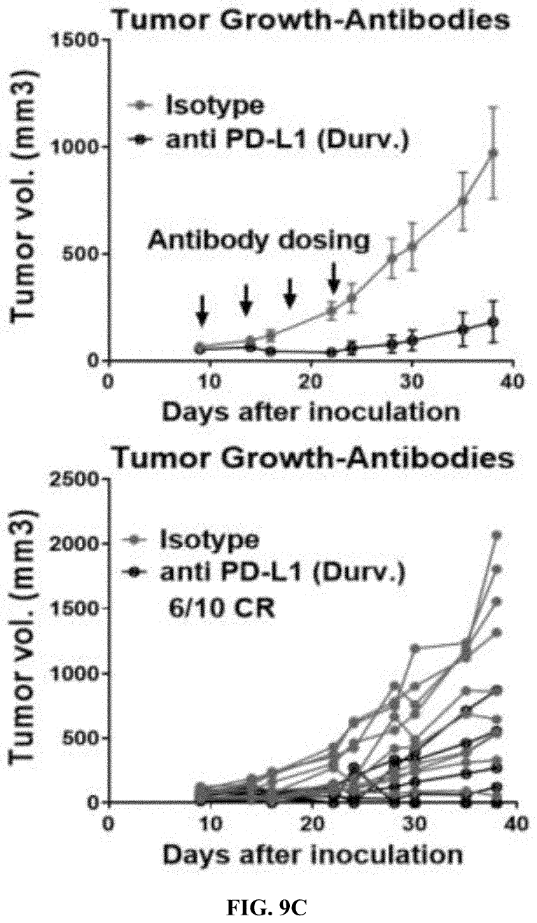

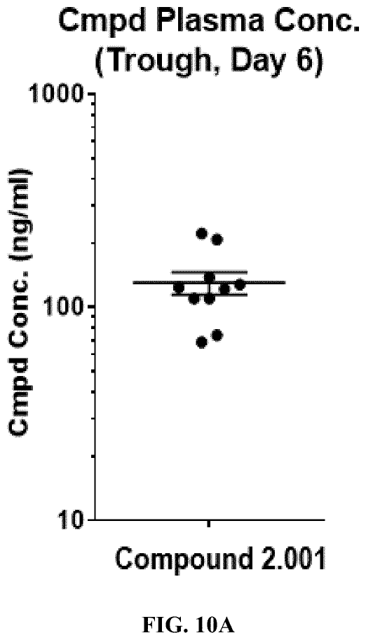

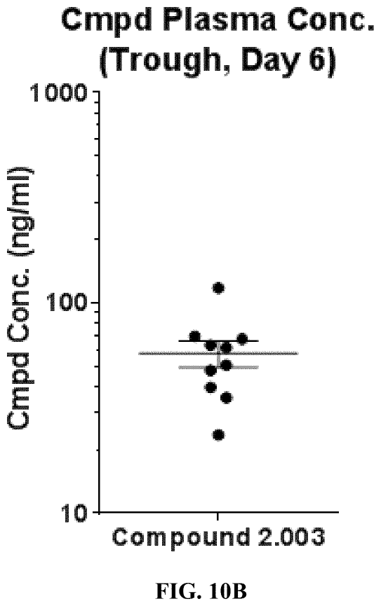

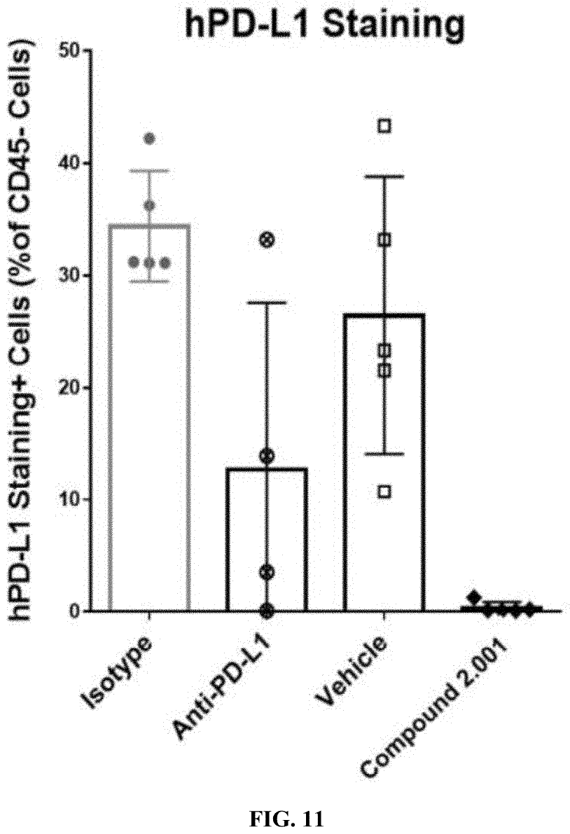

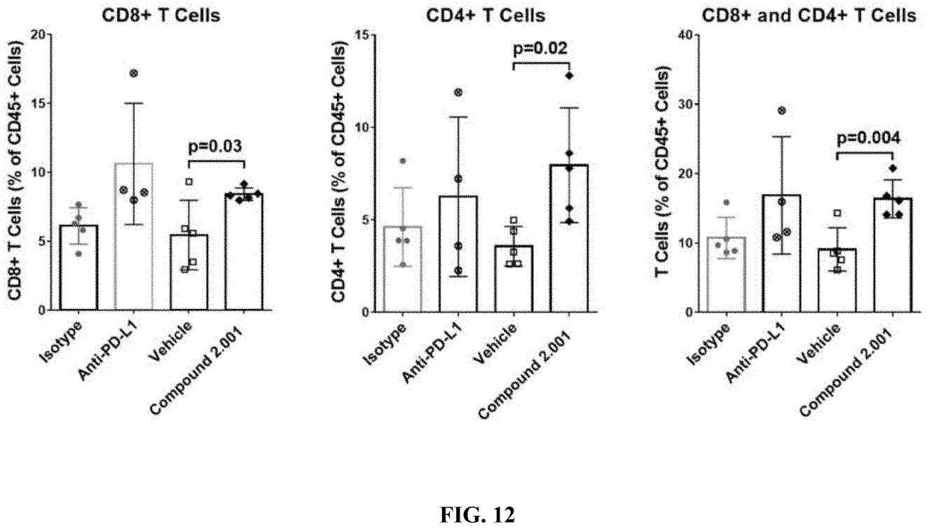

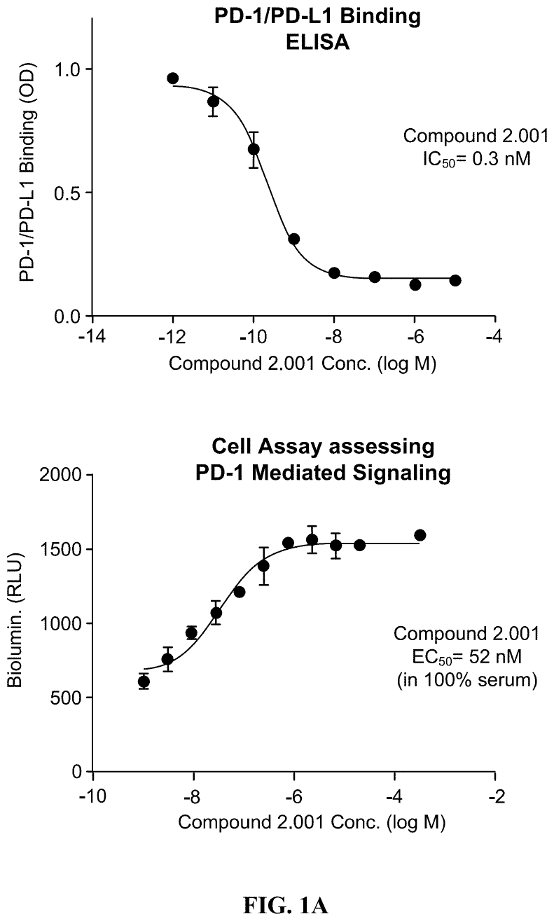

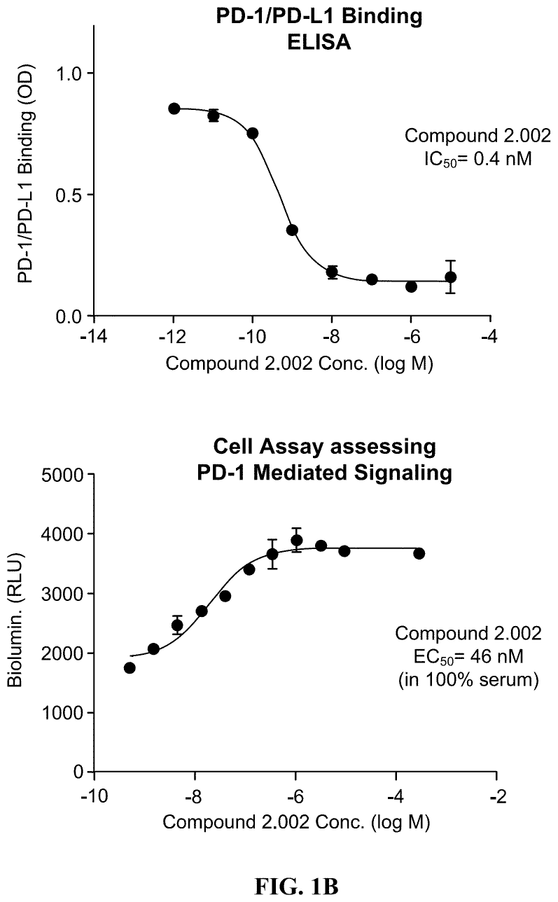

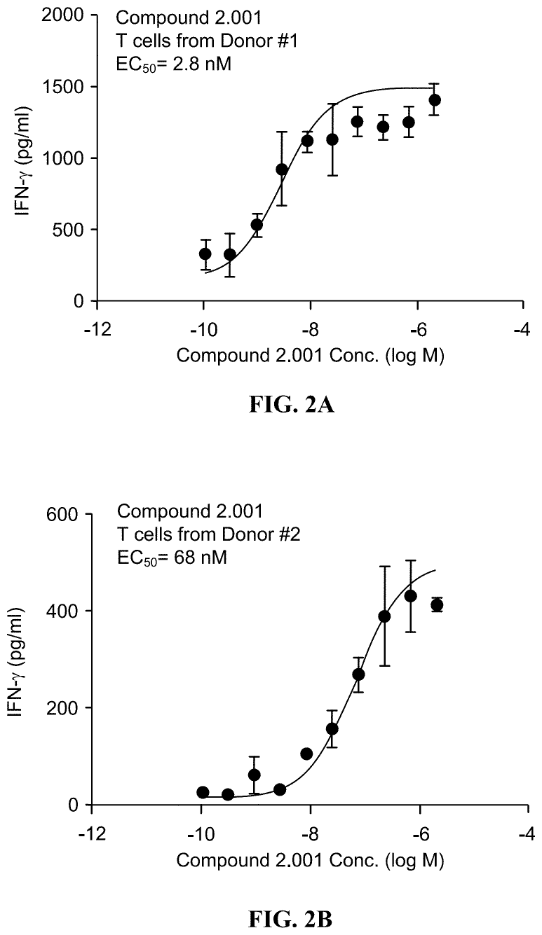

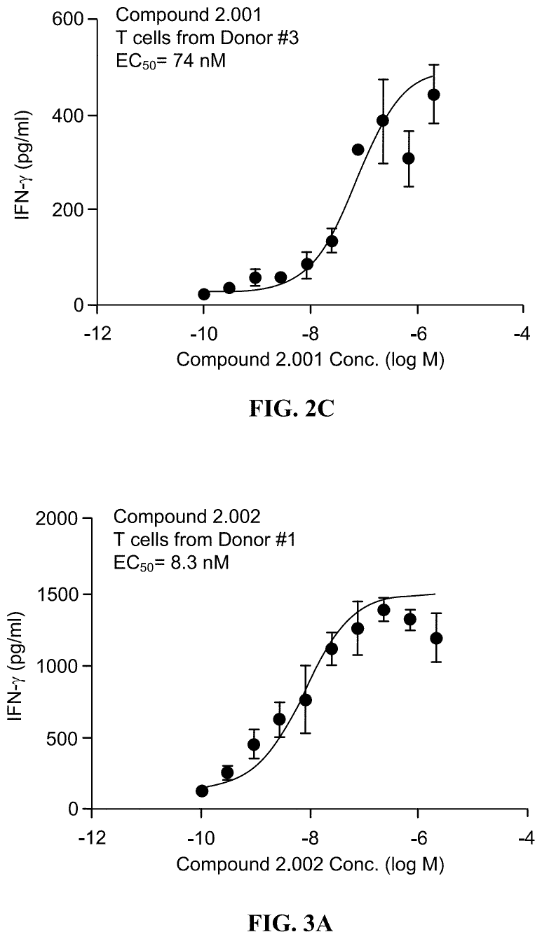

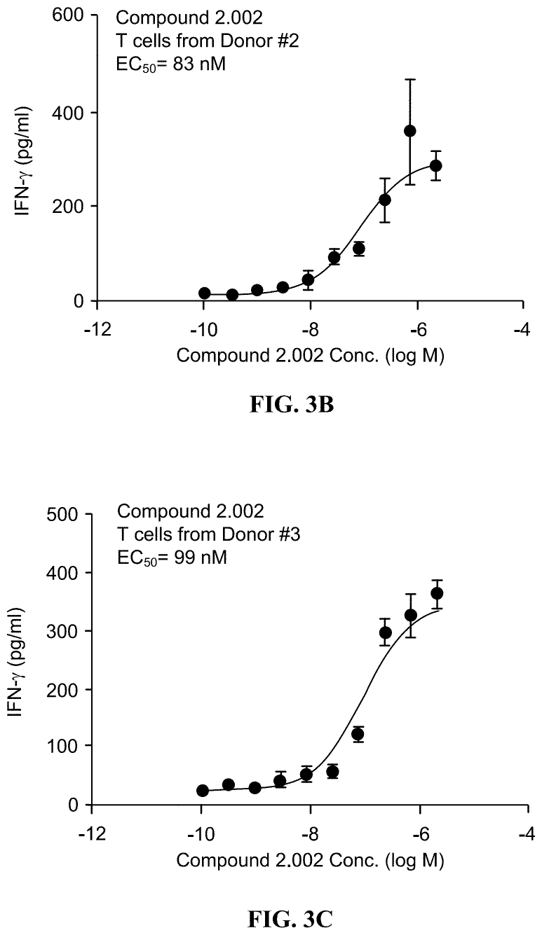

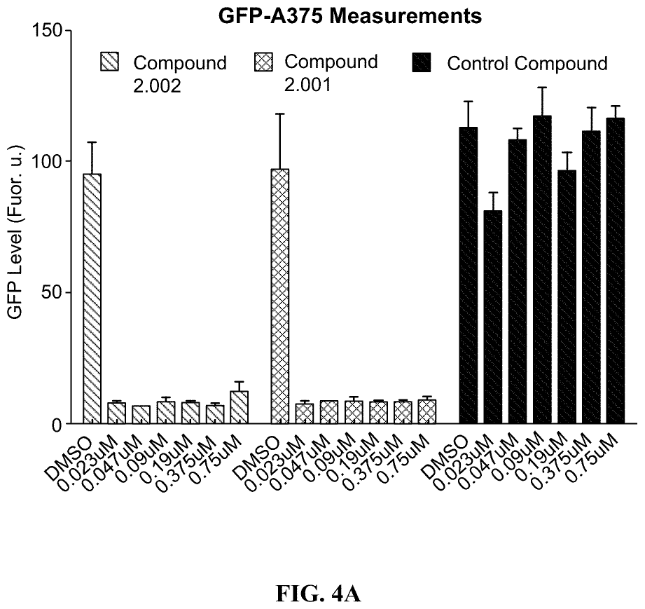

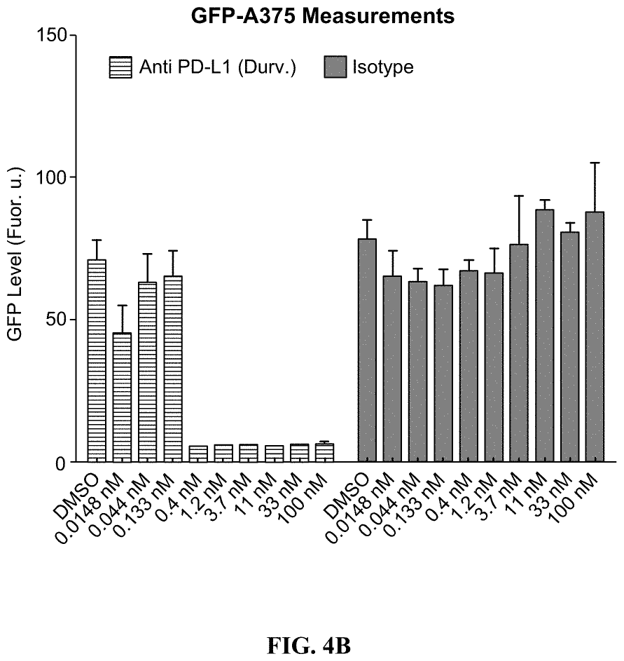

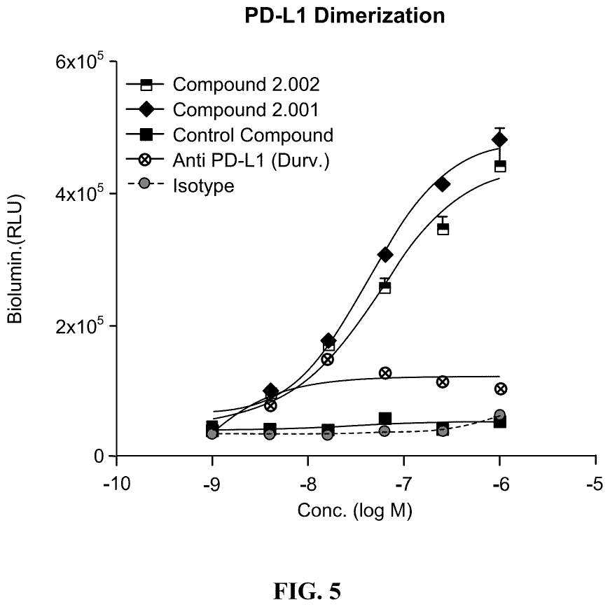

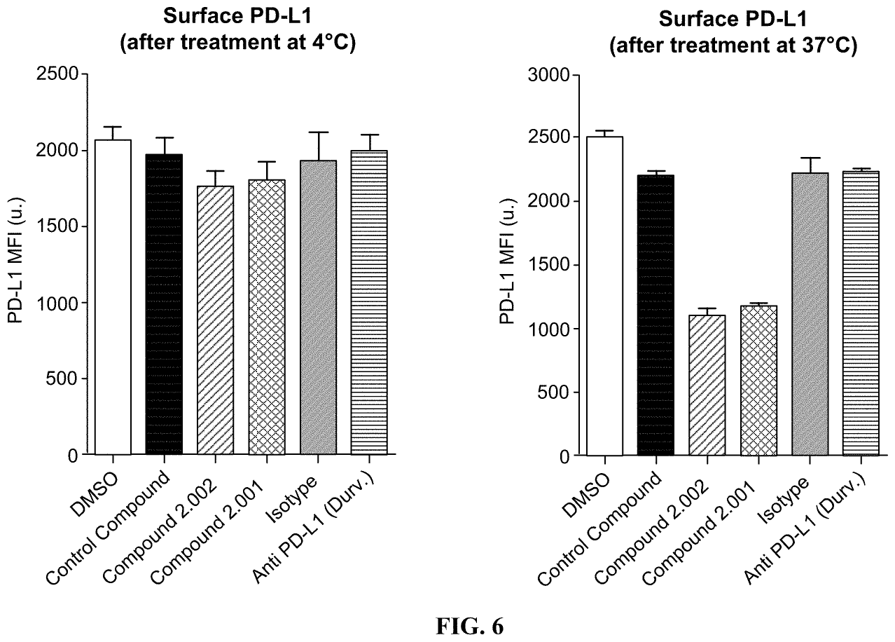

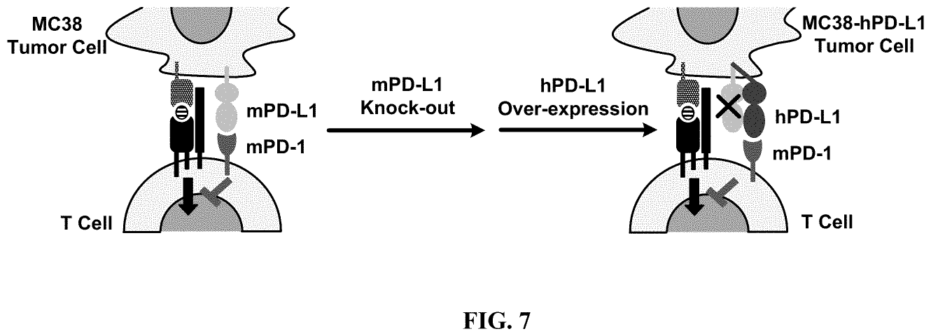

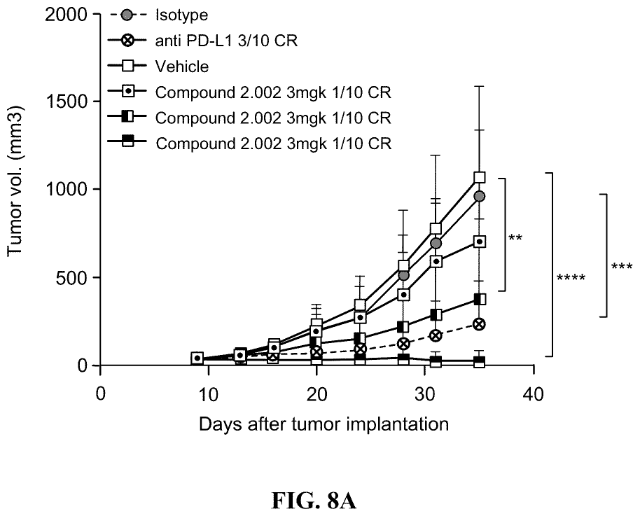

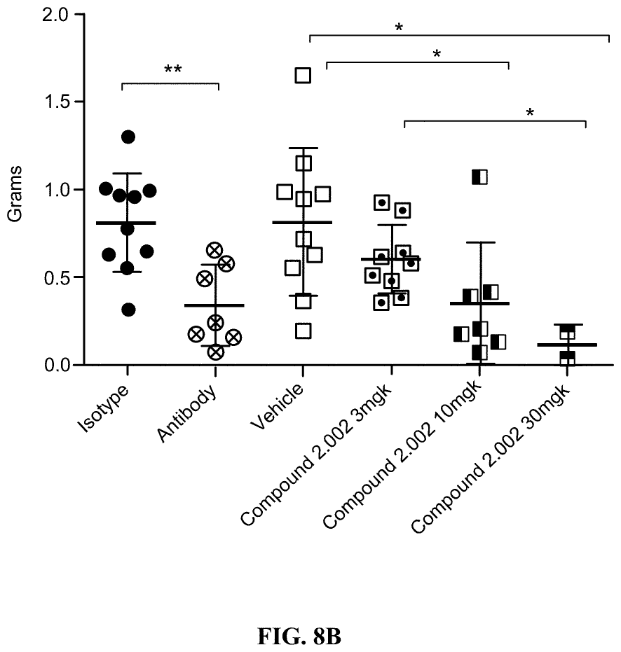

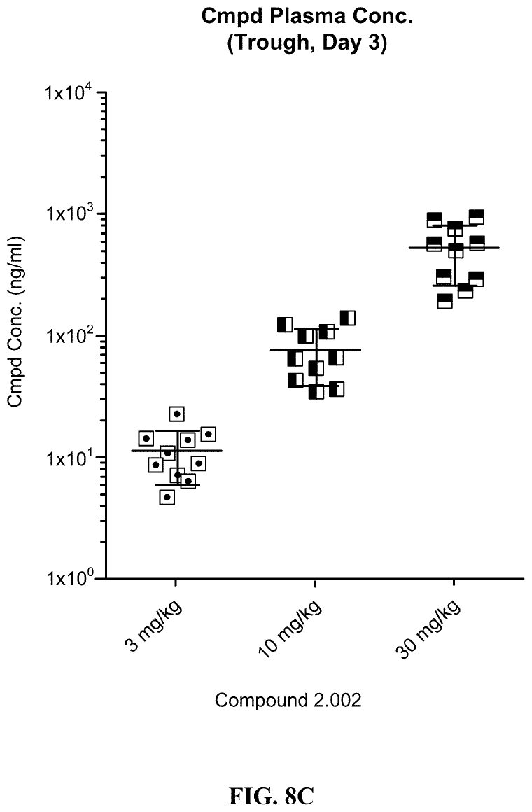

In this model, Compound 2.002, dosed orally, dose dependently suppresses tumor growth (FIG. 8A-C). Eight of the ten mice treated with 30 mg/kg (b.i.d.) Compound 2.002 achieved complete eradication of the tumors (FIG. 8A). Final tumor weights are consistent with tumor size measurements, and the eradicated tumors were not included on the tumor weight graph (FIG. 8B). Plasma compound concentrations also demonstrated dose-dependency (FIG. 8C).

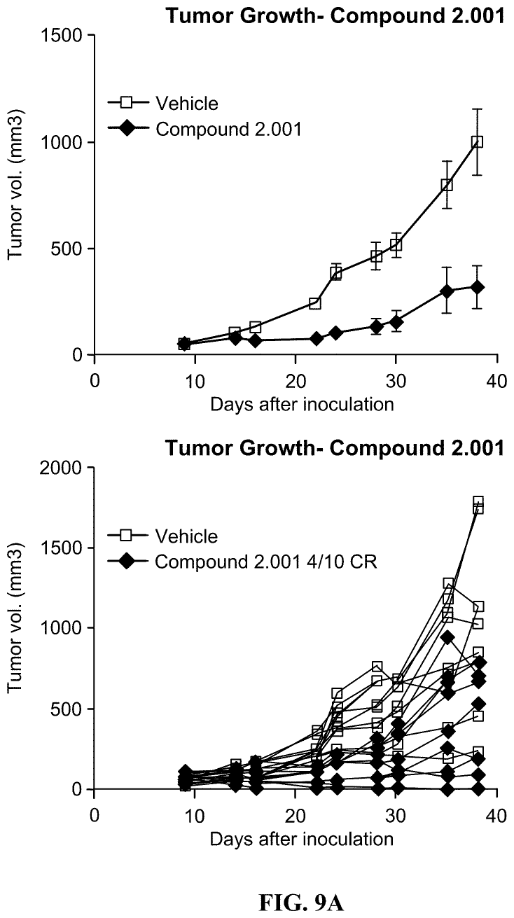

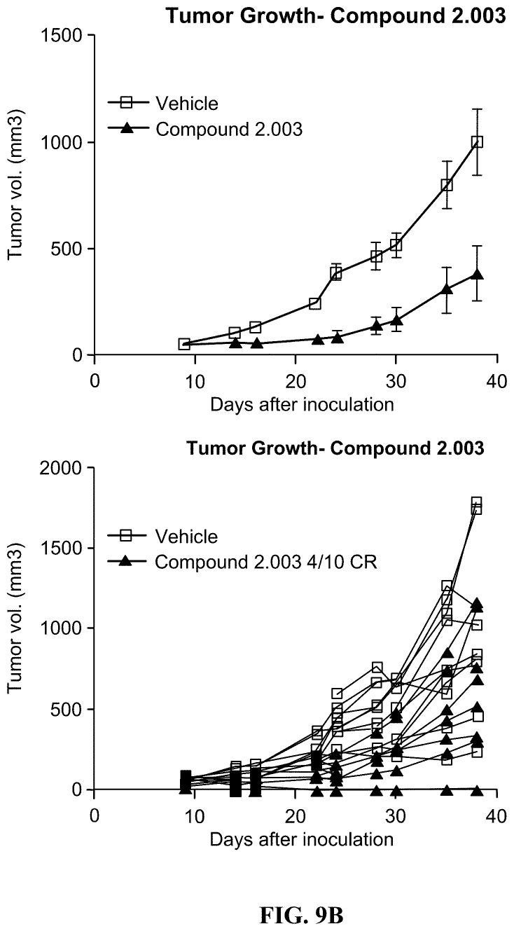

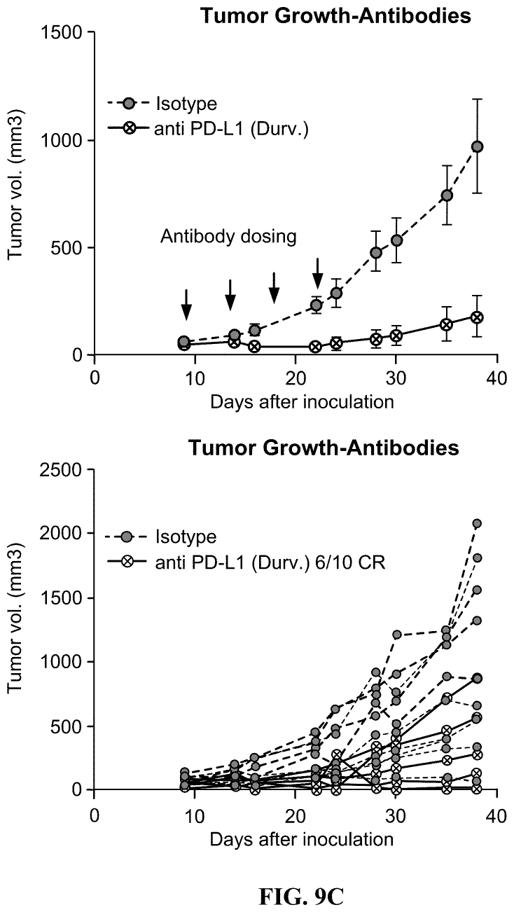

Compound 2.001, and Compound 2.003, each dosed at 30 mg/kg orally (b.i.d.), also led to similar tumor suppression as anti-PD-L1 antibody (Durvalumab) (Compare, FIG. 9A, FIG. 9B & FIG. 9C). FIG. 9A plots tumor growth when mice are administered Compound 2.001, FIG. 9B plots tumor growth when mice are administered Compound 2.003, and FIG. 9C plots tumor growth when mice are administered anti-PD-L1 antibody (Durvalumab). The top panel in each figure are the average tumor sizes from 10 mice in each group, and bottom graphs are tumor progressions of individual animals. 6 animals in anti-PD-L1 treated group, 4 in Compound 2.001 treated group, and 4 in Compound 2.001 treated group, achieved complete regression.

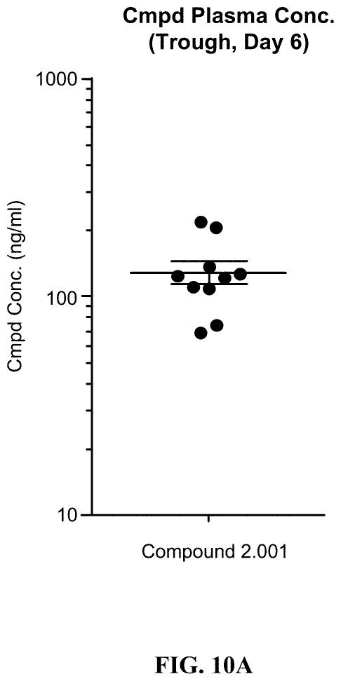

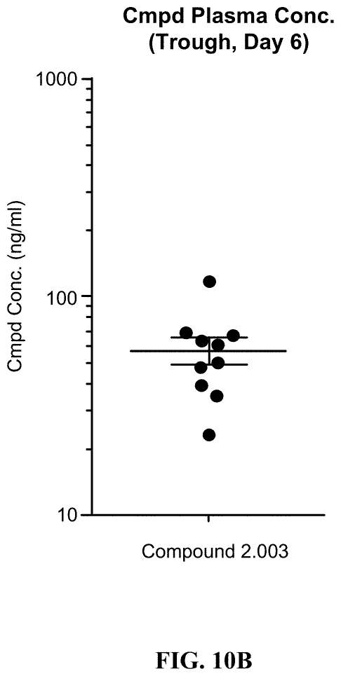

In the above referenced model, the plasma concentration for Compound 2.001 and Compound 2.003 (each dosed at 30 mg/kg, orally, b.i.d.) was measured in each mouse after 6 days of dosing. The trough plasma concentration is plotted in FIG. 10.

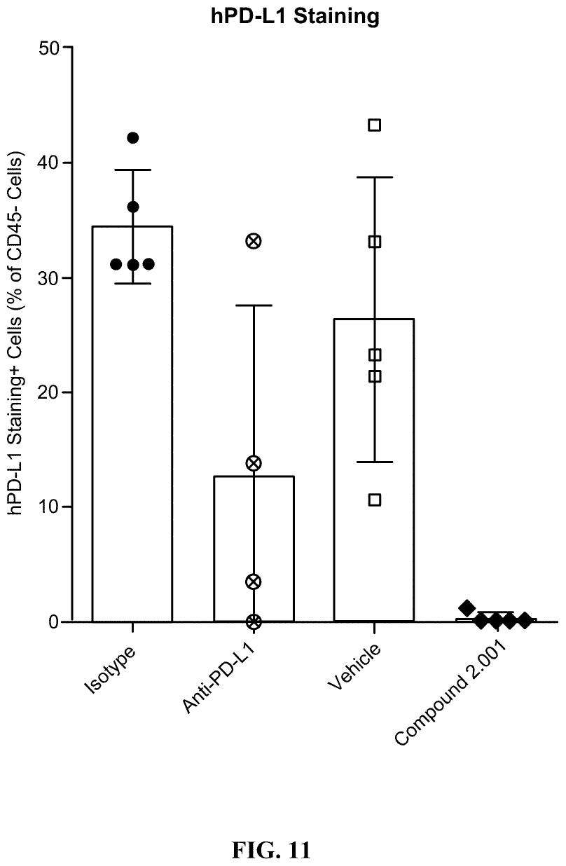

To examine the extent Compound 2.001 occupys PD-L1 on tumor cells, we utilized another PD-L1 detection antibody to stain the cells isolated from these tumors. This detection antibody does not bind to PD-L1 once Compound 2.001 or the treatment anti-PD-L1 (Durvalumab) binds. Cells from Compound 2.001 treated tumors completely lack PD-L1 staining by this detection antibody, demonstrating near complete PD-L1 occupancy by Compound 2.001 (FIG. 11).

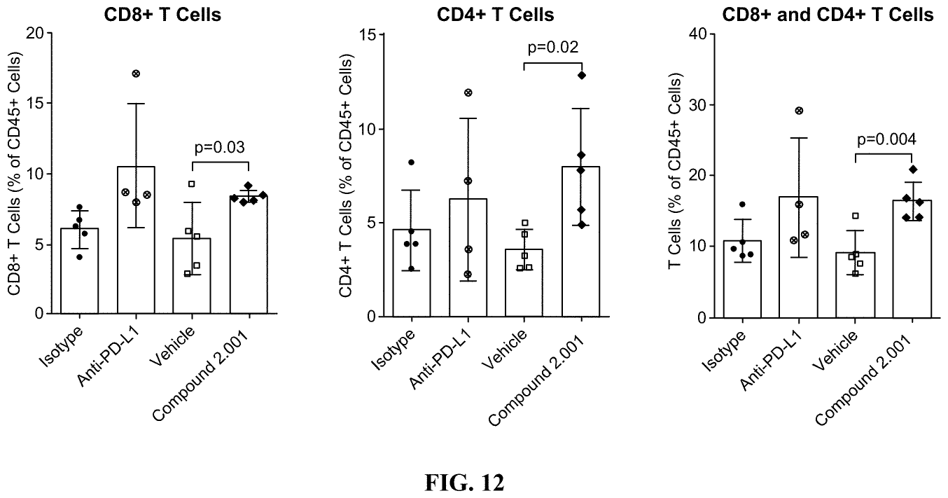

Each treatment condition in the above-referenced mouse model was analyzed for tumor infiltrating immune cells. Both CD8⁺ and CD4⁺ T cells are increased by Compound 2.001 treatment, similar to anti-PD-L1 treated tumors (FIG. 12).

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

1. A method of treating a cancer selected from the group consisting of colon cancer, renal cancer, colorectal cancer, gastric cancer, bladder cancer, melanoma, non-small cell lung cancer, Merkel cell carcinoma, liver cancer, breast cancer, and cancer of the head or neck comprising administering to a subject in need thereof an effective amount of a compound of Formula (I): 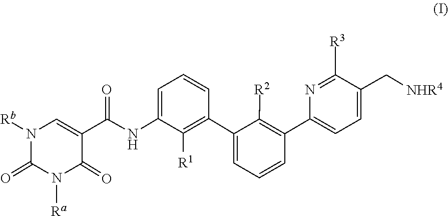
or a pharmaceutically acceptable salt thereof, wherein:
R¹ and R² are each independently selected from the group consisting of F, Cl, CH₃, and CF₃;
R³ is selected from the group consisting of F, Cl, CH₃, CF₃, —O—CH₃, and —O—CF₃;
R⁴ is selected from the group consisting of —Y and —X¹—Y wherein each X¹ is C₁₋₄ alkylene and, and Y is selected from the group consisting of C₃₋₆ cycloalkyl, C₄₋₆ heterocycloalkyl having 1 to 3 heteroatom ring vertices independely selected from the group consisting of N, O, and S, and 5- to 6-membered heteroaryl having 1 to 3 heteroatom ring vertices independently selected from the group consisting of N, O, and S, each of which is unsubstituted or substituted with one to two substituents independently selected from the group consisting of oxo, OH, C₁₋₄ alkyl, C₁₋₄ haloalkyl, C₁₋₄ hydroxyalkyl, C₁₋₄ alkoxy, C₁₋₄haloalkoxy, and C₁₋₄ hydroxyalkoxy; and
R^{a} and R^{b} are independently selected from the group consisting of H, C₁₋₃ alkyl, and C₁₋₄ haloalkyl.

2. The method of claim 1, wherein the effective amount of a compound of Formula (I) is administered orally.

3. The method of claim 1, wherein R¹ is selected from the group consisting of Cl and CH₃.

4. The method of claim 1, wherein R¹ is Cl.

5. The method of claim 1, wherein R¹ is CH₃.

6. The method of claim 1, wherein R² is selected from the group consisting of Cl and CH₃.

7. The method of claim 1, wherein R² is Cl.

8. The method of claim 1, wherein R² is CH₃.

9. The method of claim 1, wherein R³ is selected from the group consisting of —O—CH₃ and —O—CF₃.

10. The method of claim 1, wherein R³ is —O—CH₃.

11. The method of claim 1, wherein R³ is —O—CF₃.

12. The method of claim 1, wherein R^{a} is selected from the group consisting of H, CH₃, and CF₃.

13. The method of claim 1, wherein R^{a} is CH₃.

14. The method of claim 1, wherein R^{b} is selected from the group consisting of H, CH₃, and CF₃.

15. The method of claim 1, wherein R^{b} is CH₃.

16. The method of claim 1, wherein the compound of Formula I has the Formula (Ia): 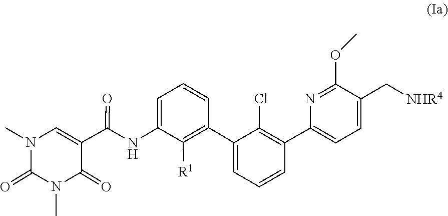
or a pharmaceutically acceptable salt thereof.

17. The method of claim 1, wherein —NH(R⁴) is selected from the group consisting of:

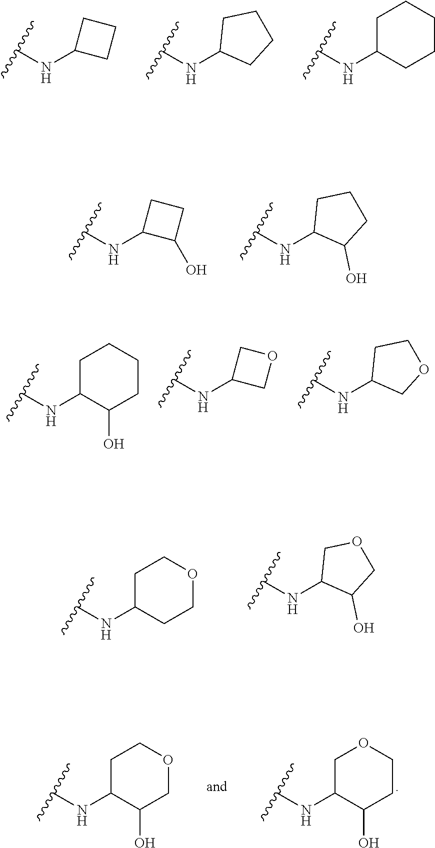

18. The method of claim 1, wherein —NH(R⁴) is selected from the group consisting of:

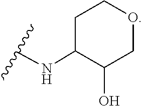 [SMILES: *NC1CCOCC1O]

19. The method of claim 1, wherein —NHR⁴ is selected from the group consisting of:

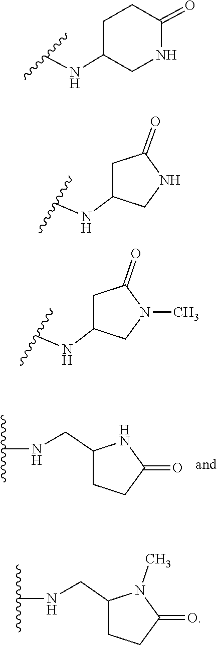

20. The method of claim 1, wherein —NHR⁴ is

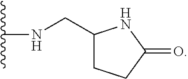 [SMILES: *NCC1CCC(=O)N1]

21. The method of claim 1, wherein the compound of Formula (I) is an optically pure or enriched isomer.

22. The method of claim 1, wherein the compound of Formula (I) is selected from a compound in Table 1.

23.-33. (canceled)
